(19) United States
(12) Patent Application Publication
(10) Pub. No.: US 2009/0169558 A1
(43) Pub. Date: Jul. 2, 2009

(54) BICYCLIC AROMATIC COMPOUNDS USEFUL AS INHIBITORS OF MITOGEN-ACTIVATED PROTEIN KINASE-ACTIVATED PROTEIN KINASE-2

(75) Inventors: Richard Heng, Hegenheim (FR); Guido Koch, Bettingen (CH); Achim Schlapbach, Lörrach (DE); Juraj Velcicky, Basel (CH); Rudolf Wälchli, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/089,335

(22) PCT Filed: Oct. 4, 2006
(86) PCT No.: PCT/EP2006/009597

§ 371 Date: Apr. 4, 2008

(30) Foreign Application Priority Data
Oct. 4, 2005 (GB) 0520164.5

(51) Int. Cl. A61K 31/519; C07D 487/04; A61K 31/52; A61K 39/395

(52) U.S. Cl. 424/141.1; 544/236; 544/117; 514/265.1; 514/263.2; 514/252.16

(57) ABSTRACT

A compound of formula (I) or a pharmaceutically acceptable salt or prodrug ester thereof:

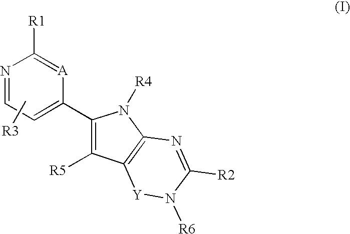

wherein the groups R1-R6, A and Y are as defined in the specification.

This application is a U.S. National Phase filing of PCT/EP2006/009597 filed 4 Oct. 2006, and claims priority to GB Patent Application 0520164.5 filed 4 Oct. 2005, the contents of which are incorporated herein by reference in their entirety.

The present invention relates to novel bicyclic aromatic compounds as inhibitors of mitogen-activated protein kinase-activated protein kinase-2 (MK2 or MAPKAP kinase-2).

Accordingly the present invention provides a compound of formula (I) or a pharmaceutically acceptable salt or a pharmaceutically-acceptable and -cleavable ester, or acid addition salt thereof

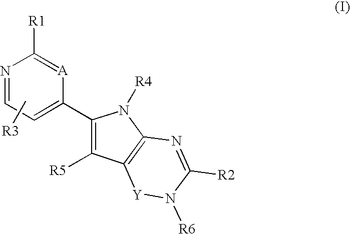

wherein A is CH or N;

Y is C═O, S═O or S(═O)₂;

R1 denotes the group —X—R¹¹;

X is a direct bond or is selected from the group consisting of C₁-C₆ alkyl, C₂-C₆ alkenyl, C₂-C₆ alkynyl, amino, aminocarbonyl, oxy, carbonyl, carboxy; carboxamido, sulfonamido, aminosulfonyl, diazo, mercapto, —CH═N—N—, —CH═N—N—CO—, —CH═N—N—CO—N—;

R¹¹ is selected from the group consisting of optionally substituted (C₁-C₆ alkyl, aryl, C₃-C₁₂ cycloalkyl, heteroaryl, heterocycloalkyl);

the optional substituent or substituents on R¹¹ being independently selected from the following: nitro, cyano, halo, hydroxyl, further optionally substituted (aryl, cycloalkyl, heteroaryl, heterocycloalkyl, aryl-C₁-C₆ alkyl, heteroaryl-C₁-C₆ alkyl, heterocycloalkyl-C₁-C₆ alkyl, cycloalkyl-C₁-C₆ alkyl, C₁-C₆ alkyl, C₂-C₆ alkenyl, C₂-C₆ alkynyl, C₁-C₆ alkyloxy, amino, carbamoyl, C₁-C₆ alkoxy, oxy, carboxy, mercapto, carboxamido, sulfonyl, sulfonamido);

such further optional substituents being selected from the group consisting of C₁-C₆ alkyl, cycloalkyl, heterocycloalkyl, aryl, heteroaryl, halo, cyano, nitro, alkoxy, hydroxyl, further optional substituted (amino, oxy, carboxy, mercapto, carboxy, carboxamido, sulfonyl, sulfonamide, alkanoyloxy;

further optional substituents being selected from the group consisting of C₁-C₆ alkyl, aryl, heteroaryl, halo, cyano, nitro, alkoxy, amino, alkylamino, dialkylamino, carboxyl, C₁-C₆ alkylcarboxyl;

R2 is selected from the group consisting of H, halo, cyano, optionally substituted (C₁-C₆ alkyl, C₂-C₆ alkenyl, C₃-C₁₂ cycloalkyl, aryl, heteroaryl, amino, mercapto, alkoxy);

the optional substituents on R2 being selected from C₁-C₆ alkyl, cycloalkyl, carboxy, sulfonyl, halo, cyano, hydroxy, alkoxy, oxy and amino.

R3 is selected from the group consisting of H, optionally substituted (C₁-C₆ alkyl, amino, alkoxy), halo, cyano and hydroxyl, optional substituents being halo, hydroxyl, alkoxy, C₁-C₆ alkyl or an amino group;

R4 is selected from the group consisting of H, optionally substituted C₁-C₆ alkyl; the optional substituent on R4 being independently selected from: halo, cyano, C₁-C₆ alkyl, amino, alkylamino, dialkylamino, hydroxyl, alkoxy, carboxy, carboxamido;

R5 is selected from the group consisting of H, halo, cyano, optionally substituted (C₁-C₆ alkyl, amino, alkoxy);

wherein the optional substituent is/are independently selected from the list as defined for R4;

R6 is selected from the group consisting of H or optionally substituted (C₁-C₄ alkyl or C₂-C₄ alkenyl) wherein the optional substituent or substituents are independently selected from one or more of the following: halo, CN, OH, OR, NHR, NR₂, SO₂NHR, SO₂NR₂, CO₂H, CO₂R, CONHR, CONH₂, CONR₂, PO₃H₂, PO₃R₂; R denoting a C₁-C₆ alkyl group.

The present invention further provides a compound of formula (I′) or a pharmaceutically acceptable salt or a pharmaceutically-acceptable and -cleavable ester, or acid addition salt thereof

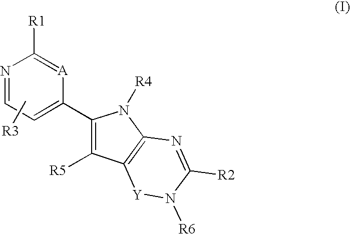

wherein A is CH or N;

Y is C═O, S═O or S(═O)₂;

R1 denotes the group —X—R¹¹;

X is a direct bond or is selected from the group consisting of C₁-C₆ alkyl, C₂-C₆ alkenyl, C₂-C₆ alkynyl, amino, aminocarbonyl, oxy, carbonyl, carboxy; carboxamido, sulfonamido, aminosulfonyl, diazo, mercapto, —CH═N—N—, —CH═N—N—CO—, —CH═N—N—CO—N—;

R¹¹ is selected from the group consisting of optionally substituted (C₁-C₆ alkyl, aryl, C₃-C₁₂ cycloalkyl, heteroaryl, heterocycloalkyl);

the optional substituent or substituents on R¹¹ being independently selected from the following: nitro, cyano, halo, hydroxyl, further optionally substituted (aryl, cycloalkyl, heteroaryl, heterocycloalkyl, C₁-C₆ alkyl, C₂-C₆ alkenyl, C₂-C₆ alkynyl, C₁-C₆ alkyloxy, amino, oxy, carboxy, mercapto, carboxamido, sulfonyl, sulfonamido);

such further optional substituents being selected from the group consisting of C₁-C₆ alkyl, cycloalkyl, heterocycloalkyl, halo, cyano, nitro, alkoxy, hydroxyl, further optional substituted (amino, oxy, carboxy, mercapto, carboxy, carboxamido, sulfonyl, sulfonamide;

further optional substituents being selected from the group consisting of C₁-C₆ alkyl, aryl, heteroaryl, halo, cyano, nitro, alkoxy, amino, alkylamino, dialkylamino;

R2 is selected from the group consisting of H, halo, cyano, optionally substituted (C₁-C₆ alkyl, C₂-C₆ alkenyl, C₃-C₁₂ cycloalkyl, aryl, heteroaryl, amino, mercapto, alkoxy);

the optional substituents on R2 being selected from C₁-C₆ alkyl, cycloalkyl, carboxy, sulfonyl, halo, cyano, hydroxy, alkoxy, oxy and amino.

R3 is selected from the group consisting of H, optionally substituted (C₁-C₆ alkyl, amino, alkoxy), halo, cyano and hydroxyl, optional substituents being halo, hydroxyl, alkoxy, C₁-C₆ alkyl or an amino group;

R4 is selected from the group consisting of H, optionally substituted C₁-C₆ alkyl; the optional substituent on R4 being independently selected from: halo, cyano, C₁-C₆ alkyl, amino, alkylamino, dialkylamino, hydroxyl, alkoxy, carboxy, carboxamido;

R5 is selected from the group consisting of H, halo, cyano, optionally substituted (C₁-C₆ alkyl, amino, alkoxy);

wherein the optional substituent is/are independently selected from the list as defined for R4;

R6 is selected from the group consisting of H or optionally substituted (C₁-C₄ alkyl or C₂-C₄ alkenyl) wherein the optional substituent or substituents are independently selected from one or more of the following: halo, CN, OH, OR, NHR, NR₂, SO₂NHR, SO₂NR₂, CO₂H, CO₂R, CONHR, CONH₂, CONR₂, PO₃H₂, PO₃R₂; R denoting a C₁-C₆ alkyl group.

Preferably, in compounds of formula (I) or (I′), X is C₂-C₆ alkenyl which may be substituted. More preferably, X is substituted ethylenyl. Alternatively preferably, X is substituted amino or is a direct bond.

Preferably, R¹¹ is aryl or heteroaryl. For example, R¹¹ may be optionally substituted (phenyl, pyridinyl, quinolinyl or indolyl).

R2 is preferably selected from H, cyano, halo or optionally substituted (amino, mercapto, alkoxy, methyl, ethyl and propyl). More preferably it is H.

R3 is preferably H, halo or optionally substituted C₁-C₆ alkyl.

R4 is preferably H.

R5 is preferably H.

R6 is preferably H.

A second aspect of the invention provides a compound of formula (II) or a pharmaceutically acceptable salt or a pharmaceutically-acceptable and -cleavable ester, or acid addition salt thereof:

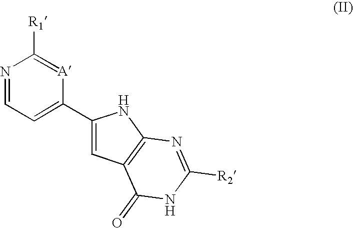

wherein A′ is CH or N;

R₁′ denotes the group —X′—R^{11′};

X is a direct bond or is selected from the group consisting of C₁-C₆ alkyl, C₂-C₆ alkenyl, C₂-C₆ alkynyl, amino, aminocarbonyl, oxy, carbonyl, carboxy; carboxamido, sulfonamido, aminosulfonyl, diazo, mercapto, —CH═N—N—, —CH═N—N—CO—, —CH═N—N—CO—N—;

R¹¹ is selected from the group consisting of optionally substituted (C₁-C₆ alkyl, aryl, C₃-C₁₂ cycloalkyl, heteroaryl, heterocycloalkyl);

the optional substituent or substituents on R¹¹ being independently selected from the following: nitro, cyano, hydroxyl, halo, further optionally substituted (aryl, cycloalkyl, heteroaryl, heterocycloalkyl, C₁-C₆ alkyl, C₂-C₆ alkenyl, C₂-C₆ alkynyl, C₁-C₆ alkyloxy, amino, oxy, carboxy, mercapto, carboxy, carboxamido, sulfonyl, sulfonamido);

such further optional substituents being selected from the group consisting of C₁-C₆ alkyl, cycloalkyl, heterocycloalkyl, halo, cyano, hydroxyl, nitro, alkoxy, further optional substituted (amino, oxy, carboxy, mercapto, carboxy, carboxamido, sulfonyl, sulfonamido); the further optional substituents being as defined above with respect to R¹¹.

Preferably A′ is CH.

Preferably X′ is C₂-C₆ alkenyl which may be substituted. More preferably, X′ is substituted ethylenyl. Alternatively preferably, X′ is a substituted amino or is a direct bond.

Preferably, R^{11′} is aryl or heteroaryl. For example, R^{11′} may be optionally substituted (phenyl, pyridinyl, quinolinyl, or indolyl).

Preferably R₂′ is H, halo, cyano or optionally substituted (amino, mercapto, alkoxy, methyl, ethyl, propyl). More preferably it is H.

A third aspect of the invention provides a compound of formula (III) or a pharmaceutically acceptable salt or a pharmaceutically-acceptable and -cleavable ester, or acid addition salt thereof:

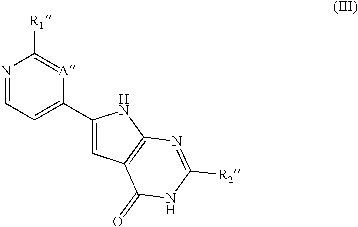

wherein A″ is CH or N;

R₁″ is selected from the following:

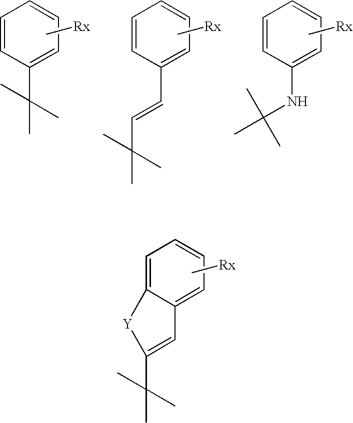

wherein:

Y is O, N, S or —C═N—;

Rx is selected from optionally substituted (aryl, cycloalkyl, heteroaryl, heterocycloalkyl, C₁-C₆ alkyl, C₂-C₆ alkenyl, C₂-C₆ alkynyl, C₁-C₆ alkyloxy, amino, oxy, carboxy, mercapto, carboxamido, sulfonyl, sulfonamido), hydroxyl, halo, nitro, cyano;

the optional substituents on Rx being selected from the group consisting of C₁-C₆ alkyl, cycloalkyl, heterocycloalkyl, halo, cyano, hydroxyl, amino, alkylamino, dialkylamino, carboxy, carboxamido, sulfonamido;

R₂″ is selected from H, halo, cyano, optionally substituted (C₁-C₆ alkyl, C₂-C₆ alkenyl, C₃-C₁₂ cycloalkyl, aryl, heteroaryl, mercapto, alkoxy, amino); the optional substituents being as defined above for Rx.

For the avoidance of doubt, the terms listed below are to be understood to have the following meaning throughout the present description and claims:

The term “lower”, when referring to organic radicals or compounds means a compound or radical with may be branched or unbranched with up to and including 7 carbon atoms.

A lower alkyl group may be branched, unbranched or cyclic and contains 1 to 7 carbon atoms, preferably 1 to 4 carbon atoms. Lower alkyl represents, for example: methyl, ethyl, propyl, butyl, isopropyl, isobutyl, tertiary butyl or 2,2-dimethylpropyl.

A lower alkoxy group may be branched or unbranched and contains 1 to 7 carbon atoms, preferably 1 to 6 carbon atoms. Lower alkoxy represents, for example: methoxy, ethoxy, propoxy, butoxy, isopropoxy, isobutoxy or tertiary butoxy. Lower alkoxy includes cycloalkyloxy and cycloalkyl-lower alkyloxy.

A lower alkene, alkenyl or alkenoxy group is branched or unbranched and contains 2 to 7 carbon atoms, preferably 1 to 4 carbon atoms and contains at least one carbon-carbon double bond. Lower alkene, lower alkenyl or lower alkenyloxy represents for example vinyl, prop-1-enyl, allyl, butenyl, isopropenyl or isobutenyl and the oxy equivalents thereof.

A lower alkyne or alkynyl group is branched or unbranched and contains 2 to 7 carbon atoms, preferably 1 to 4 carbon atoms and contains at least one carbon-carbon triple bond. Lower alkyne or lower alkynyl or lower alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo.

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one to three heteroatoms. Heterocyclic aryl represents, for example: pyridyl, indolyl, quinoxalinyl, quinolinyl, isoquinolinyl, benzothienyl, benzofuranyl, benzothiophenyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl, benzthiazolyl, benzoxazolyl, Heterocyclic aryl also includes such substituted radicals.

Cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted. Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. The term heterocycloalkyl is intended also to include bridged heterocycloalkyl groups such as 3-hydroxy-8-aza-bicyclo[3.2.1]oct-8-yl or 2,6-diazatricyclo[3.3.1.1*3,7*]dec-1-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxymaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Preferred compounds of formula (I) are:
6-[2-((E)-Styryl)-pyridin-4-yl]-3,7-dihydro-pyrrolo[2,3-d]pyrimidin-4-one
6-[2-((E)-Styryl)-pyridin-4-yl]-2-trifluoromethyl-3,7-dihydro-pyrrolo[2,3-d]pyrimidin-4-one
2-Methyl-6-[2-((E)-styryl)-pyridin-4-yl]-3,7-dihydro-pyrrolo[2,3-d]pyrimidin-4-one
2-Amino-6-[2-((E)-styryl)-pyridin-4-yl]-3,7-dihydro-pyrrolo[2,3-d]pyrimidin-4-one
6-[2-(4-Fluoro-phenyl)-pyridin-4-yl]-3,7-dihydro-pyrrolo[2,3-d]pyrimidin-4-one
6-[2-(3-Fluoro-phenyl)-pyridin-4-yl]-3,7-dihydro-pyrrolo[2,3-d]pyrimidin-4-one
6-[2-(3-Amino-phenyl)-pyridin-4-yl]-3,7-dihydro-pyrrolo[2,3-d]pyrimidin-4-one
6-[2-(3-Fluoro-4-methoxy-phenyl)-pyridin-4-yl]-3,7-dihydro-pyrrolo[2,3-d]pyrimidin-4-one
6-(6′-Methoxy-[2,3′]bipyridinyl-4-yl)-3,7-dihydro-pyrrolo[2,3-d]pyrimidin-4-one
6-[2-(4-Morpholin-4-yl-phenyl)-pyridin-4-yl]-3,7-dihydro-pyrrolo[2,3-d]pyrimidin-4-one
6-[2-(4-Morpholin-4-ylmethyl-phenyl)-pyridin-4-yl]-3,7-dihydro-pyrrolo[2,3-d]pyrimidin-4-one
6-{2-[(E)-2-(4-Fluoro-phenyl)-vinyl]-pyridin-4-yl}-3,7-dihydro-pyrrolo[2,3-d]pyrimidin-4-one
6-(2-Benzofuran-2-yl-pyridin-4-yl)-3,7-dihydro-pyrrolo[2,3-d]pyrimidin-4-one
6-{2-[(E)-2-(4-Dimethylamino-phenyl)-vinyl]-pyridin-4-yl}-3,7-dihydro-pyrrolo[2,3-d]pyrimidin-4-one
2-Amino-6-{2-[(E)-2-(4-dimethylamino-phenyl)-vinyl]-pyridin-4-yl}-3,7-dihydro-pyrrolo[2,3-d]pyrimidin-4-one
2-Amino-6-{2-[(E)-2-(4-fluoro-phenyl)-vinyl]-pyridin-4-yl}-3,7-dihydro-pyrrolo[2,3-d]pyrimidin-4-one
6-(2-Benzo[b]thiophen-2-yl-pyridin-4-yl)-3,7-dihydro-pyrrolo[2,3-d]pyrimidin-4-one
6-(2-Quinolin-3-yl-pyridin-4-yl)-3,7-dihydro-pyrrolo[2,3-d]pyrimidin-4-one
6-[2-(1H-Indol-2-yl)-pyridin-4-yl]-3,7-dihydro-pyrrolo[2,3-d]pyrimidin-4-one
6-(2-{(E)-2-[4-(4-Methyl-piperazin-1ylmethyl)-phenyl]-vinyl}-pyridin-4-yl)-3,7-dihydro-pyrrolo[2,3-d]pyrimidine-one
6-{2-[(E)-2-(4-Diethylaminomethyl-phenyl)-vinyl]-pyridin-4-yl}-3,7-dihydro-pyrrolo[2,3-d]pyrimidin-4-one
2,2-Dimethyl-propionic acid 4-{(E)-2-[4-(4-oxo-4,7-dihydro-3H-pyrrolo[2,3-d]pyrimidin-6yl)-pyridin-2-yl]-vinyl}-benzyl ester
2,2-Dimethyl-propionic acid 2-{(E)-2-[4-(4-oxo-4,7-dihydro-3H-pyrrolo[2,3-d]pyrimidin-6yl)-pyridin-2-yl]-vinyl}-benzyl ester
6-{2-[(E)-2-(4-Piperidin-1-ylmethyl-phenyl)-vinyl]-pyridin-4-yl}-3,7-dihydro-pyrrolo[2,3-d]pyrimidin-4-one
4-(4-{(E)-2-[4-(4-Oxo-4,7-dihydro-3H-pyrrolo[2,3-d]pyrimidin-6-yl)-pyridin-2-yl]-vinyl}-benzyl)-piperazine-1-carboxylic acid tert.-butyl ester
6-{2-[(E)-2-(3-Fluoro-4-morpholin-4-ylmethyl-phenyl)-vinyl]-pyridin-4-yl}-3,7-dihydro-pyrrolo[2,3-d]pyrimidin-4-one
6-{2-[-2-(3-Fluoro-4-morpholin-4-ylmethyl-phenyl)-ethyl]-pyridin-4-yl}-3,7-dihydro-pyrrolo[2,3-d]pyrimidin-4-one
6-{2-[(E)-2-(3-Morpholin-4-ylmethyl-phenyl)-vinyl]-pyridin-4-yl}-3,7-dihydro-pyrrolo[2,3-d]pyrimidin-4-one
6-(2-{(E)-2-[4-(2-Morpholin-4-yl-2-oxo-ethyl)-phenyl]-vinyl}-pyridin-4-yl)-3,7-dihydro-pyrrolo[2,3-d]pyrimidin-4-one
6-[2-(4-Morpholin-4-ylmethyl-phenylethynyl)-pyridin-4-yl]-3,7-dihydro-pyrrolo[2,3-d]pyrimidin-4-one
6-{2-[2-(4-Morpholin-4-ylmethyl-phenyl)ethyl]-pyridin-4-yl}-3,7-dihydro-pyrrolo[2,3-d]pyrimidine-4-one
6-{2-[(E)-2-(4-Morpholin-4-ylmethyl-phenyl)-vinyl]-pyridin-4-yl}-3,7-dihydro-pyrrolo[2,3-d]pyrimidin-4-one
N,N-Diethyl-4-{(E)-2-[4-(4-oxo-4,7-dihydro-3H-pyrrolo[2,3-d]pyrimidin-6-yl)-pyridin-2-yl]-vinyl}-benzamide
6-(2-{(E)-2-[4-(Morpholine-4-carbonyl)-phenyl]-vinyl}-pyridin-4-yl)-3,7-dihydro-pyrrolo[2,3-d]pyrimidin-4-one
6-(2-{(E)-2-[4-(4-Hydroxy-piperidine-1-carbonyl)-phenyl]-vinyl}-pyridin-4-yl)-3,7-dihydro-pyrrolo[2,3-d]pyrimidin-4-one
6-(2-{(E)-2-[3-(Morpholine-4-carbonyl)phenyl]-vinyl}-pyridin-4-yl)-3,7-dihydro-pyrrolo[2,3-d]pyrimidin-4-one
6-[2-((E)-2-Pyridin-3-yl-vinyl)-pyridin-4-yl]-3,7-dihydro-pyrrolo[2,3-d]pyrimidin-4-one
6-(2-{(E)-2-[4-(4-Acetyl-piperazin-1-ylmethyl)-phenyl]-vinyl}-pyridin-4-yl)-3,7-dihydro pyrrolo[2,3-d]pyrimidin-4-one
2-Amino-6-{2-[(E)-2-(4-morpholin-4-ylmethyl-phenyl)-vinyl]-pyridin-4-yl}-3,7-dihydro-pyrrolo[2,3-d]pyrimidin-4-one
6-{2-[(E)-2-(4-Morpholin-4-ylmethyl-phenyl)-vinyl]-pyridin-4-yl}-2-trifluoromethyl-3,7-dihydro-pyrrolo[2,3-d]pyrimidin-4-one
2-Methyl-6-{2-[(E)-2-(4-morpholin-4-ylmethyl-phenyl)-vinyl]-pyridin-4-yl}-3,7-dihydro-pyrrolo[2,3-d]pyrimidin-4-one
6-{2-[(E)-2-(4-Hydroxyphenyl)-vinyl]-pyridin-4-yl}-3,7-dihydro-pyrrolo[2,3-d]pyrimidin-4-one
6-[2-((E)-2-Cyclohexyl-vinyl)-pyridin-4-yl]-3,7-dihydro-pyrrolo[2,3-d]pyrimidin-4-one
6-(2-{(E)-2-[4-(2-Dimethylamino-ethoxy)-phenyl]-vinyl}-pyridin-4-yl)-3,7-dihydro-pyrrolo[2,3-d]pyrimidin-4-one
6-(2-{(E)-2-[4-(2-Morpholin-4-yl-ethoxy)-phenyl]-vinyl}-pyridin-4-yl)-3,7-dihydro-pyrrolo[2,3-d]pyrimidin-4-one
6-(2-{(E)-2-[4-(2-Hydroxy-2-methyl-propoxy)-phenyl]-vinyl}-pyridin-4-yl)-3,7-dihydro-pyrrolo[2,3-d]pyrimidin-4-one
6-[6′-(4-Methyl-piperazin-1-yl)-[2,3′]bipyridinyl-4-yl]-3,7-dihydro-pyrrolo[2,3-d]pyrimidin-4-one
2-Amino-6-[6′-(4-methyl-piperazin-1-yl)-[2,3′]bipyridinyl-4-yl]-3,7-dihydro-pyrrolo[2,3-d]pyrimidin-4-one
6-(6′-Pyrrolidin-1-yl-[2,3′]bipyridinyl-4-yl)-3,7-dihydro-pyrrolo[2,3-d]pyrimidin-4-one
6-[6′-(2-Pyrrolidin-1-yl-ethoxy)-[2,3′]bipyridinyl-4-yl]-3,7-dihydro-pyrrolo[2,3-d]pyrimidin-4-one
2-Benzyl-6-[2-((E)-styryl)-pyridin-4-yl]-3,7-dihydro-pyrrolo[2,3-d]pyrimidin-4-one
2-Butyl-6-[2-((E)-styryl)-pyridin-4-yl]-3,7-dihydro-pyrrolo[2,3-d]pyrimidin-4-one
2-Cyclopropyl-6-[2-((E)-styryl)-pyridin-4-yl]-3,7-dihydro-pyrrolo[2,3d]pyrimidin-4-one
2-Methylsulfanyl-6-[2-((E)-styryl)-pyridin-4-yl]-3,7-dihydro-pyrrolo[2,3-d]pyrimidin-4-one
2-(2,3-Dihydroxy-propylamino)-6-[2-((E)-styryl)-pyridin-4-yl]-3,7-dihydro-pyrrolo[2,3-d]pyrimidin-4-one
2-(2-Hydroxy-1-hydroxymethyl-ethylamino)-6-[2-((E)-styryl)-pyridin-4-yl]-3,7-dihydro-pyrrolo[2,3-d]pyrimidin-4-one
6-[2-(4-Benzyloxy-phenyl)-pyridin-4-yl]-3,7-dihydro-pyrrolo[2,3-d]pyrimidin-4-one
N-Cyclopentyl-4-[4-(4-oxo-4,7-dihydro-3H-pyrrolo[2,3-d]pyrimidin-6-yl)-pyridin-2-yl]-benzamide
N-(4-Hydroxy-cyclohexyl)-4-[4-(4-oxo-4,7-dihydro-3H-pyrrolo[2,3-d]pyrimidin-6-yl)-pyridin-2-yl]-benzamide
N-[2-(2-Methoxy-ethoxy)-ethyl]-4-[4-(4-oxo-4,7-dihydro-3H-pyrrolo[2,3-d]pyrimidin-6-yl)-pyridin-2-yl]-benzamide
2-(3-Methyl-butylamino)-6-[2-((E)-styryl)-pyridin-4-yl]-3,7-dihydro-pyrrolo[2,3-d]pyrimidin-4-one
2-(2-Hydroxy-ethylamino)-6-[2-((E)-styryl)-pyridin-4-yl]-3,7-dihydro-pyrrolo[2,3-d]pyrimidin-4-one
3-(2-Methoxy-ethyl)-6-[2-((E)-styryl)-pyridin-4-yl]-3,7-dihydro-pyrrolo[2,3-d]pyrimidin-4-one.

The invention in a fourth aspect provides a compound of formula (I), (II) or (III) or a pharmaceutically-acceptable and -cleavable ester, or acid addition salt thereof for use as a pharmaceutical.

The invention in a fifth aspect provides the use of a compound of formula (I), (II) or (III) or a pharmaceutically-acceptable and -cleavable ester, or acid addition salt thereof in the manufacture of a medicament for the treatment of an autoimmune disease or condition.

The invention in a sixth aspect provides the use of a compound of formula (I), (II) or (III) or a pharmaceutically-acceptable and -cleavable ester, or acid addition salt thereof for the treatment of cytokine mediated, e.g. TNF alpha mediated and/or MK2 related conditions.

The invention in a seventh aspect provides a method of treatment of cytokine mediated, e.g. TNF alpha mediated and/or MK2 related conditions comprising administering an effective amount of a compound of formula (I), (II) or (III) or a pharmaceutically-acceptable and -cleavable ester, or acid addition salt thereof to a patient in need of such treatment.

The invention in an eighth aspect provides a pharmaceutical composition comprising a compound of formula (I), (II) or (III) or a pharmaceutically-acceptable and -cleavable ester, or acid addition salt thereof in association with a pharmaceutically acceptable excipient, diluent or carrier.

In a ninth aspect the invention provides a process for preparing a compound of formula (I) in free or salt form, comprising the step of:

(i) reacting a compound of formula X with a compound of formula XIa or XIb:

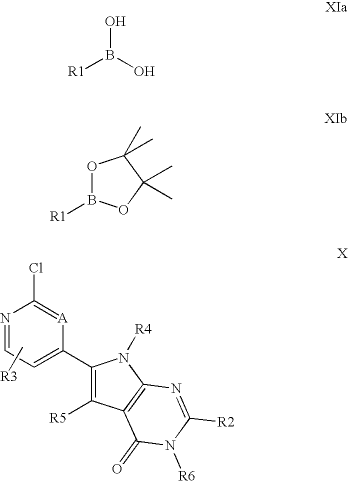

in the presence of a suitable catalyst, a base and solvent; R1-R6, A and Y being as defined above with respect to formula (I). A suitable catalyst for this reaction is for example PdCl₂(PPh₃)₂ in the presence of Na₂CO₃ in n-propanol/water as solvent. An alternative convenient catalyst for this reaction is PdCl₂(dppf)₂ in the presence of Na₂CO₃ in DMF/water; or

(ii) for compounds of formula (I) wherein R1 is a substituted amino group having the formula R¹¹—NH—, by reacting a compound of formula X with a compound of formula XIII:

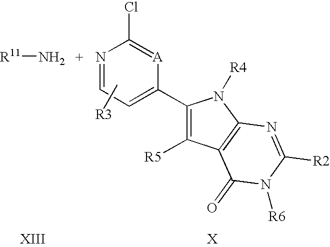

Using a suitable catalyst in the presence of a base and solvent. A convenient catalyst is Pd₂ dba₃/phosphine ligand, e.g. DPEphos in the presence of a base such as NaOtBu in a solvent such as dioxane; or

(iii) for compounds of formula (I) wherein A is N and R1 is denoted by R¹¹—X— wherein X is a direct bond, by reacting any compound of formula XV with a suitable organometallic reagent R¹¹-M:

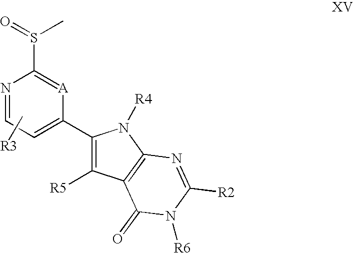

using a suitable anhydrous solvent, for example THF. Suitable organometallic reagents include Grignard reagents, e.g. R¹¹—MgBr; or

(iv) for compounds of formula I wherein A is N and R1 is R¹¹—NH—, by reacting a compound of formula XV as shown above with a compound of formula R¹¹—NH₂ wherein R11 is defined above with respect to formula (i), in the presence of a base and a suitable solvent. A suitable base is n-BuLi in THF.

The compounds of formula I in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

The compound of formula X may be prepared by the following reaction scheme:

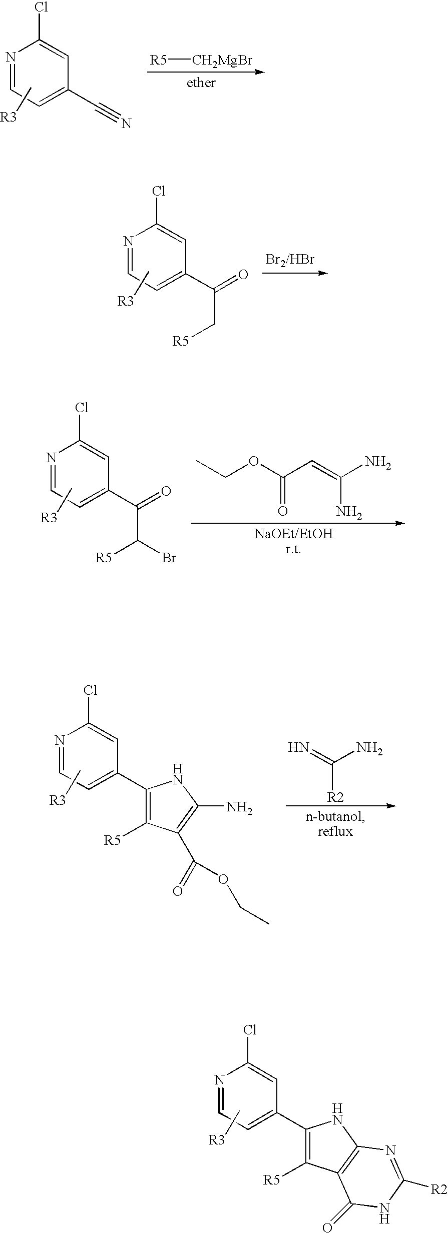

Compounds of formula XV may be prepared by the following reaction scheme:

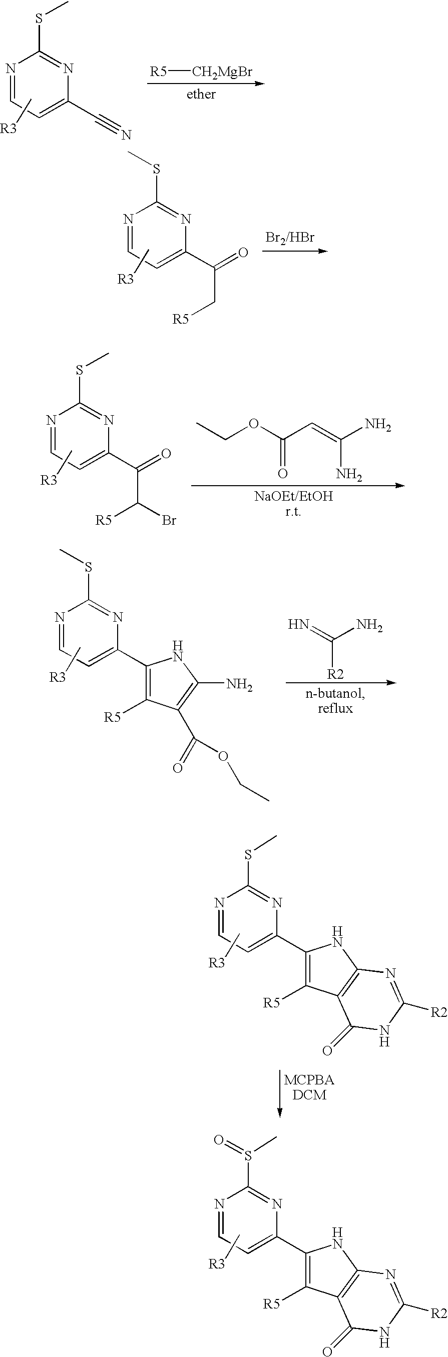

Additionally, compounds of formula I can be prepared by the following scheme:

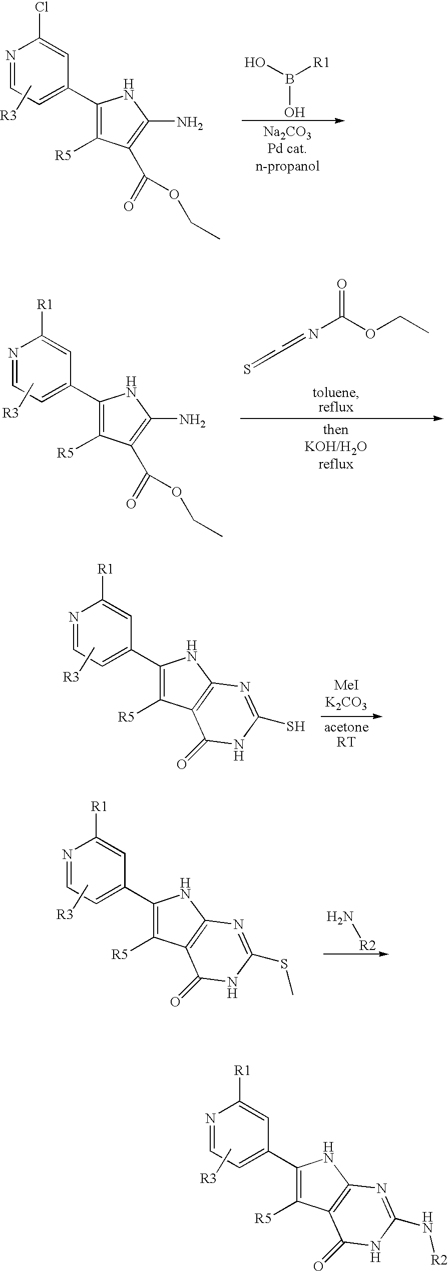

Agents of the invention may be prepared by processes described below, which are intended to be non-limiting examples:

### EXAMPLE 1

### 6-[2-((E)-Styryl)-pyridin-4-yl]-3,7-dihydro-pyrrolo[2,3-d]pyrimidin-4-one

### a) 2-Bromo-1-(2-chloro-pyridin-4-yl)-ethanone Hydrobromide

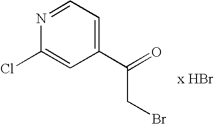

2-Bromo-1-(2-chloro-pyridin-4-yl)-ethanone hydrobromide is synthesised as outlined in WO 2004/058762. Crystallisation from ether gives the title product as off white solid.

¹H-NMR (400 MHZ, DMSO-d6): 5.02 (s, 2H), 7.84 (d, 1H), 7.98 (s, 1H), 8.66 (d, 1H).

MS (ESI⁺) m/z: 234 (80%), 236 (100%), 238 (25%) [MH]⁺

### b) 2-Amino-5-(2-chloro-pyridin-4-yl)-1H-pyrrole-3-carboxylic acid Ethyl Ester

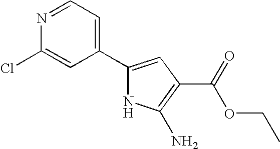

2-Bromo-1-(2-chloro-pyridin-4-yl)ethanone hydrobromide (3.0 g, 12.8 mmol) is stirred in a mixture of 35 ml aqueous NaHCO₃ solution and ether to liberate the free base. The aqueous phase is extracted two more times with ether, the ether phase dried and concentrated. Carbamimidoyl-acetic acid ethyl ester hydrochloride (4.26 g, 25.6 mmol) (Liebigs Ann. Chem. 1977, 1895) is suspended in 10 ml ethanol and cooled to 0° C. To this mixture sodium ethoxide (1.74 g, 25.6 mmol) is added. The mixture is stirred for 20 minutes, then 2-bromo-1-(2-chloro-pyridin-4-yl)ethanone in 10 ml ethanol is added dropwise. After stirring at r.t. for 16 hours the reaction is stopped by adding 100 ml water and the mixture is extracted with ethylacetate. The material obtained after removal of the solvent is used for further steps without purification.

¹H-NMR (400 MHZ, DMSO-d6): 1.27 (t, 3H), 4.16 (q, 2H), 5.97 (s, 2H), 6.98 (d, 1H), 7.44 (dd, 1H), 7.55 (d, 1H), 8.14 (d, 1H), 11.1 (s, 1H).

MS (ESI⁺) m/z: 266 [MH]⁺

### c) 6-(2-Chloro-pyridin-4-yl)-3,7-dihydro-pyrrolo[2,3-d]pyrimidin-4-one

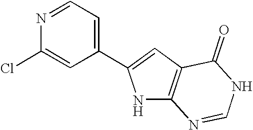

2-Amino-5-(2-chloro-pyridin-4-yl)-1H-pyrrole-3-carboxylic acid ethyl ester (500 mg, 1.9 mmol) and formamidine hydrochloride (758 mg, 9.4 mmol) are refluxed in 12 ml n-butanol for 30 hours. NaHCO3 solution is added, the mixture extracted with ethylacetate, dried and the solvent evaporated. Tituration of the solid residue with ether gives the desired product.

¹H-NMR (400 MHZ, DMSO-d6): 7.38 (s, 1H), 7.81 (d, 1H), 7.93 (s, 1H), 7.95 (s, 1H), 8.34 (d, 1H), 11.7-12.5 (br, 2H).

MS (ESI⁺) m/z: 247 [MH]⁺

### d) 6-[2-((E)-Styryl)-pyridin-4-yl]-3,7-dihydro-pyrrolo[2,3-d]pyrimidin-4-one

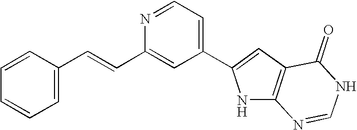

E-phenyl-vinyl boronic acid (150 mg, 0.61 mmol) and 6-(2-Chloro-pyridin-4-yl)-3,7-dihydro-pyrrolo[2,3-d]pyrimidin-4-one (247 mg, 0.61 mmol) are dissolved in 4 ml n-propanol/2N Na2CO3 (4:1). The solution is degassed by introduction of a stream of argon, Pd(PPh₂)₂Cl₂ (20 mg, 0.03 mmol) is added and the mixture is heated under reflux for 3 hours. The reaction is quenched with saturated NaHCO₃ solution, extracted into ethylacetate, the organic phase is dried over Na₂SO₄ and the solvent is evaporated. Purification by reverse phase HPLC (Waters X-Terra, acetonitrile/water) yields the title compound.

¹H-NMR (400 MHZ, DMSO-d6): 7.25 (d, 1H), 7.29 (s, 1H), 7.33 (t, 1H), 7.41 (t, 2H), 7.61-7.67 (m, 3H), 7.71 (d, 1H), 7.91 (s, 1H), 8.04 (s, 1H), 8.51 (d, 1H), 12.5 (brs, 1H). one NH signal obscured.

MS (ESI⁺) m/z: 315 [MH]⁺

### EXAMPLE 2

### 6-[2-((E)-Styryl)-pyridin-4-yl]-2-trifluoromethyl-3,7-dihydro-pyrrolo[2,3-d]pyrimidin-4-one

### a) 6-(2-Chloro-pyridin-4-yl)-2-trifluoromethyl-3,7-dihydro-pyrrolo[2,3-d]pyrimidin-4-one

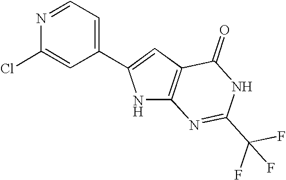

The title compound is prepared as described in Example 1c) starting from 400 mg 2-amino-5-(2-chloro-pyridin-4-yl)-1H-pyrrole-3-carboxylic acid ethyl ester and 1.12 g trifluoroacetamidine. The product is obtained as white solid.

¹H-NMR (400 MHZ, DMSO-d6): 7.08 (s, 1H), 7.67 (d, 1H), 7.78 (s, 1H), 8.24 (d, 1H), 11.9 (s, 1H), one NH obscured.

MS (ESI⁺) m/z: 315 [MH]⁺

### b) 6-[2-((E)-Styryl)-pyridin-4-yl]-2-trifluoromethyl-3,7-dihydro-pyrrolo[2,3-d]pyrimidin-4-one

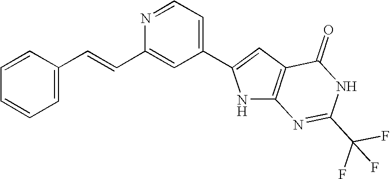

The title compound is prepared in analogy to Example 1d).

¹H-NMR (400 MHZ, DMSO-d6): 6.99 (s, 1H), 7.22 (d, 1H), 7.30 (t, 1H), 7.40 (t, 2H), 7.54 (d, 1H), 7.65 (d, 2H), 7.70 (d, 1H), 7.92 (s, 1H), 8.43 (d, 1H), 11.80 (s, 1H), pyrrol NH not visible.

MS (ESI⁺) m/z: 383 [MH]⁺

### EXAMPLE 3

### 2-Methyl-6-[2-((E)-styryl)-pyridin-4-yl]-3,7-dihydro-pyrrolo[2,3-d]pyrimidin-4-one

### a) 6-(2-Chloro-pyridin-4-yl)-2-methyl-3,7-dihydro-pyrrolo[2,3-d]pyrimidin-4-one

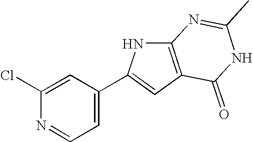

A mixture of 2-amino-5-(2-chloro-pyridin-4-yl)-1H-pyrrole-3-carboxylic acid ethyl ester (500 mg, 1.9 mmol), ethyl acetamidate hydrochloride (581 mg, 4.7 mmol) and sodium ethylate 320 mg, 4.7 mmol) in 10 ml dimethyl-acetamide is stirred at 120 C for 36 hours. The reaction mixture is poured onto aq. sodium bicarbonate solution and extracted with ethylacetate. The organic phases are dried and concentrated. HPLC purification gives the title compound.

¹H-NMR (400 MHZ, DMSO-d6): 2.35 (s, 3H), 7.35 (s, 1H), 7.78 (d, 1H), 7.91 (s, 1H), 8.34 (d, 1H), 11.9 (s, 1H), 12.5 (s, 1H).

MS (ESI⁺) m/z: 261 [MH]⁺

### b) 2-Methyl-6-[2-((E)-styryl)-pyridin-4-yl]-3,7-dihydro-pyrrolo[2,3-d]pyrimidin-4-one

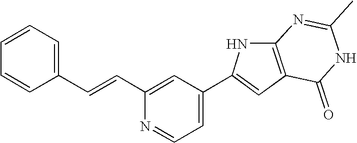

The title compound is prepared via Suzuki coupling in analogy to Example 1d).

MS (ESI⁺) m/z: 329 [MH]⁺

### EXAMPLE 4

### 2-Amino-6-[2-((E)-styryl)-pyridin-4-yl]-3,7-dihydro-pyrrolo[2,3-d]pyrimidin-4-one

### a) 2-Amino-6-(2-chloro-pyridin-4-yl)-3,7-dihydro-pyrrolo[2,3-d]pyrimidin-4-one

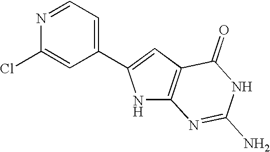

To a solution of 2,6-diamino-4-hydroxypyrimidine (269 mg, 2.1 mmol) and sodium ethoxide (159 mg, 2.3 mmol) in 8 ml ethanol is added 2-bromo-1-(2-chloro-pyridin-4-yl)ethanone (500 mg, 2.1 mmol) (free base prepared as described in example 1b). The mixture is heated under reflux for 16 hours and then quenched by addition of water. The white precipitate which forms is filtered and titurated with ether to give the target molecule.

¹H-NMR (400 MHZ, DMSO-d6): 6.34 (s, 2H), 7.17 (s, 1H), 7.65 (d, 1H), 7.77 (s, 1H), 8.22 (d, 1H), 10.41 (brs, 1H), 11.7 (brs, 1H).

MS (ESI⁺) m/z: 262 [MH]⁺

### b) 2-Amino-6-[2-((E)-styryl)-pyridin-4-yl]-3,7-dihydro-pyrrolo[2,3-d]pyrimidin-4-one

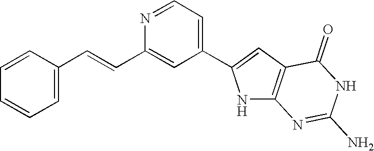

The title compound is prepared as described in Example 1d).

¹H-NMR (400 MHZ, DMSO-d6): 6.28 (s, 2H), 7.08 (d, 1H), 7.22 (d, 1H), 7.32 (t, 1H), 7.41 (t, 2H), 7.53 (dd, 1H), 7.64 (d, 2H), 7.69 (d, 1H), 7.90 (s, 1H), 8.44 (d, 1H), 10.38 (brs, 1H), 11.7 (s, 1H).

MS (ESI⁺) m/z: 330 [MH]⁺

The following compounds are synthesised in analogous manner:

### EXAMPLE 5

### 6-[2-(4-Fluoro-phenyl)-pyridin-4-yl]-3,7-dihydro-pyrrolo[2,3-d]pyrimidin-4-one

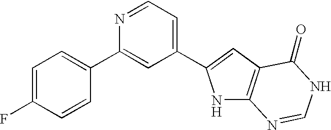

¹H-NMR (400 MHZ, DMSO-d6): 7.33 (t, 2H), 7.41 (s, 1H), 7.75 (d, 1H), 7.91 (s, 1H), 8.24 (dd, 2H), 8.39 (s, 1H), 8.58 (d, 1H), 11.9 (brs, 1H), 12.6 (brs, 1H).

MS (ESI⁻) m/z: 305 [M−H]⁻

### EXAMPLE 6

### 6-[2-(3-Fluoro-phenyl)-pyridin-4-yl]-3,7-dihydro-pyrrolo[2,3-d]pyrimidin-4-one

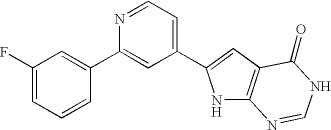

¹H-NMR (400 MHZ, DMSO-d6): 7.28 (t, 1H), 7.46 (s, 1H), 7.56 (dd, 1H), 7.81 (d, 1H), 7.93 (s, 1H), 8.00 (d, 1H), 8.06 (d, 1H), 8.45 (s, 1H), 8.61 (d, 1H), 11.8 (brs, 1H), 12.6 (brs, 1H).

MS (ESI⁺) m/z: 307 [MH]⁺

### EXAMPLE 7

### 6-[2-(3-Amino-phenyl)-pyridin-4-yl]-3,7-dihydro-pyrrolo[2,3-d]pyrimidin-4-one

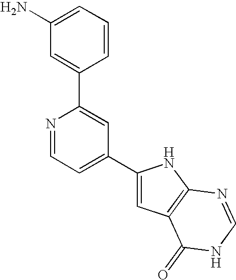

¹H-NMR (400 MHZ, DMSO-d6): 5.16 (s, 2H), 6.64 (d, 1H), 7.14 (t, 1H), 7.28-7.37 (m, 2H), 7.43 (s, 1H), 7.70 (d, 1H), 7.90 (s, 1H), 8.26 (s, 1H), 8.56 (d, 1H), 11.9 (br s, 1H), 12.6 br s, 1H).

MS (ESI⁺) m/z: 304 [MH]⁺

### EXAMPLE 8

### 6-[2-(3-Fluoro-4-methoxy-phenyl)-pyridin-4-yl]-3,7-dihydro-pyrrolo[2,3-d]pyrimidin-4-one

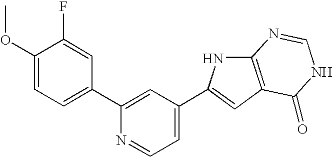

¹H-NMR (400 MHZ, DMSO-d6): 3.91 (s, 3H), 7.29 (t, 1H), 7.44-7.57 (m, 3H), 7.73 (d, 1H), 8.02 (s, 1H), 8.37 (s, 1H), 8.56 (d, 1H), 11.9 (s, 1H), 12.6 (s, 1H).

MS (ESI⁺) m/z: 337 [MH]⁺

### EXAMPLE 9

### 6-(6′-Methoxy-[2,3′]bipyridinyl-4-yl)-3,7-dihydro-pyrrolo[2,3-d]pyrimidin-4-one

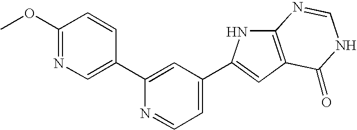

¹H-NMR (400 MHZ, DMSO-d6): 3.94 (s, 3H), 6.96 (d, 1H), 7.44 (s, 1H), 7.74 (d, 1H), 7.94 (s, 1H), 8.39 (s, 1H), 8.46 (d, 1H), 8.61 (d, 1H), 8.98 (s, 1H), 11.7 (br s, 1H), 12.1 (br s, 1H).

MS (ESI⁺) m/z: 320 [MH]⁺

### EXAMPLE 10

### 6-[2-(4-Morpholin-4-yl-phenyl)-pyridin-4-yl]-3,7-dihydro-pyrrolo[2,3-d]pyrimidin-4-one

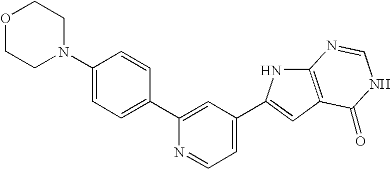

¹H-NMR (400 MHZ, DMSO-d6): 3.32-3.45 (m, 4H), 3.74 (t, 4H), 7.03 (d, 2H), 7.35 (s, 1H), 7.62 (d, 1H), 7.91 (s, 1H), 8.08 (d, 2H), 8.29 (s, 1H), 8.52 (d, 1H), 2 N—H signals obscured.

MS (ESI⁺) m/z: 374 [MH]⁺

### EXAMPLE 11

### 6-[2-(4-Morpholin-4-ylmethyl-phenyl)-pyridin-4-yl]-3,7-dihydro-pyrrolo[2,3-d]pyrimidin-4-one

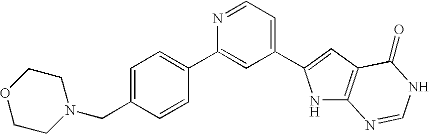

¹H-NMR (400 MHZ, DMSO-d6): 2.40 (t, 4H), 3.54 (s, 2H), 3.60 (t, 4H), 7.41 (s, 1H), 7.44 (d, 2H), 7.76 (dd, 1H), 7.94 (s, 1H), 8.17 (d, 2H), 8.40 (d, 1H), 8.60 (d, 1H), 11.95 (s, 1H), 12.70 (s, 1H).

MS (ESI⁺) m/z: 388 [MH]⁺

### EXAMPLE 12

### 6-{2-[(E)-2-(4-Fluoro-phenyl)-vinyl]-pyridin-yl}-3,7-dihydro-pyrrolo[2,3-d]pyrimidin-4-one

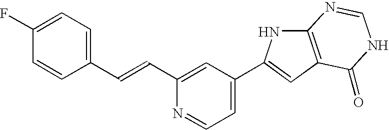

¹H-NMR (400 MHZ, DMSO-d6): 7.21 (d, 1H), 7.27 (t, 2H), 7.30 (s, 1H), 7.68 (dd, 1H), 7.71 (dd, 2H), 7.72 (d, 1H), 8.02 (s, 1H), 8.51 (s, 1H), 8.53 (d, 1H), 11.9 (br s, 1H), 12.6 (br s, 1H).

MS (ESI⁺) m/z: 333 [MH]⁺

### EXAMPLE 13

### 6-(2-Benzofuran-2-yl-pyridin-4-yl)-3,7-dihydro-pyrrolo[2,3-d]pyrimidin-4-one

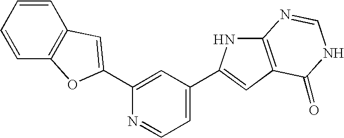

¹H-NMR (400 MHZ, DMSO-d6): 7.33 (t, 1H), 7.42 (t, 1H), 7.49 (s, 1H), 7.70 (d, 1H), 7.71 (s, 1H), 7.78 (d, 1H), 7.88 (d, 1H), 7.97 (s, 1H), 8.50 (s, 1H), 8.66 (d, 1H), 12.0 (s, 1H), 12.8 (s, 1H).

MS (ESI⁺) m/z: 329 [MH]⁺

### EXAMPLE 14

### 6-{2-[(E)-2-(4-Dimethylamino-phenyl)-vinyl]-pyridin-4-yl}-3,7-dihydro-pyrrolo[2,3-d]pyrimidin-4-one

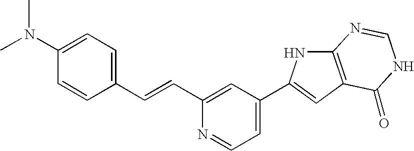

¹H-NMR (400 MHZ, DMSO-d6): 2.95 (s, 6H), 6.73 (d, 2H), 6.97 (d, 1H), 7.26 (s, 1H), 7.46 (d, 2H), 7.58 (d, 1H), 7.60 (d, 1H), 7.91 (s, 1H), 7.95 (s, 1H), 8.46 (d, 1H), 11.9 (br s, 1H), 12.5 (brs, 1H).

MS (ESI⁺) m/z: 358 [MH]⁺

### EXAMPLE 15

### 2-Amino-6-{2-[(E)-2-(4-dimethylamino-phenyl)-vinyl]-pyridin-4-yl}-3,7-dihydro-pyrrolo[2,3-d]pyrimidin-4-one

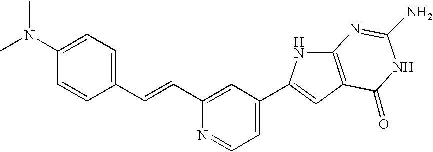

MS (ESI⁺) m/z: 373 [MH]⁺

### EXAMPLE 16

### 2-Amino-6-{2-[(E)-2-(4-fluoro-phenyl)-vinyl]-pyridin-4-yl}-3,7-dihydro-pyrrolo[2,3-d]pyrimidin-4-one

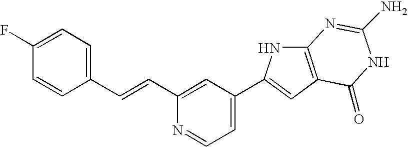

¹H-NMR (400 MHZ, DMSO-d6): 6.27 (s, 2H), 7.06 (s, 1H), 7.16 (d, 1H), 7.23 (t, 2H), 7.52 (d, 1H), 7.66 (d, 1H), 7.69 (dd, 2H), 7.88 (s, 1H), 8.43 (d, 1H), 10.4 (s, 1H), 11.7 (s, 1H).

MS (ESI⁺) m/z: 348 [MH]⁺

### EXAMPLE 17

### 6-(2-Benzo[b]thiophen-2-yl-pyridin-4-yl)-3,7-dihydro-pyrrolo[2,3-d]pyrimidin-4-one

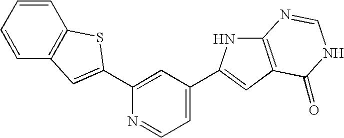

¹H-NMR (400 MHZ, DMSO-d6): 7.35-7.44 (m, 2H), 7.48 (s, 1H), 7.76 (d, 1H), 7.89 (dd, 1H), 7.95 (s, 1H), 7.98 (dd, 1H), 8.26 (s, 1H), 8.55 (d, 1H), 8.59 (s, 1H), 11.9 (s, t H), 12.6 (s, 1H).

MS (ESI⁺) m/z: 345 [MH]⁺

### EXAMPLE 18

### 6-(2-Quinolin-3-yl-pyridin-4-yl)-3,7-dihydro-pyrrolo[2,3-d]pyrimidin-4-one

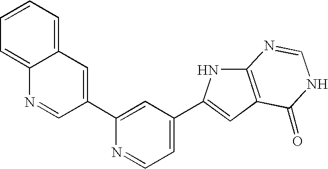

¹H-NMR (400 MHZ, DMSO-d6): 7.62 (s, 1H), 7.79 (t, 1H), 7.90-8.02 (m, 3H), 8.18 (d, 1H), 8.20 (d, 1H0, 8.76 (s, 1H), 8.78 (d, 1H), 9.33 (s, 1H), 9.78 (s, 1H), 12.0 (s, 1H), 12.8 (s, 1H).

MS (ESI⁺) m/z: 340 [MH]⁺

### EXAMPLE 19

### 6-[2-(1H-Indol-2-yl)-pyridin-4-yl]-3,7-dihydro-pyrrolo[2,3-d]pyrimidin-4-one

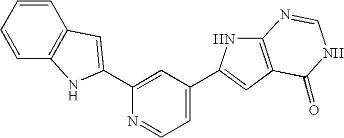

¹H-NMR (400 MHZ, DMSO-d6): 7.02 (t, 1H), 7.13 (t, 1H), 7.29 (s, 1H), 7.45 (s, 1H), 7.52-7.65 (m, 2H), 7.73 (d, 1H), 7.96 (d, 1H), 8.48 (s, 1H), 8.58 (d, 1H), 11.6 (s, 1H), 11.9 (s, 1H), 12.6 (s, 1H).

MS (ESI⁺) m/z: 328 [MH]⁺

### EXAMPLE 20

### 6-(2-{(E)-2-[4-(4-Methyl-piperazin-1-ylmethyl)-phenyl]-vinyl}-pyridin-4-yl)-3,7-dihydro-pyrrolo[2,3-d]pyrimidine-one

### a) 1-(4-Ethynyl-benzyl)-4-methyl-piperazine

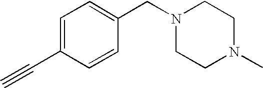

4-Ethynylbenzaldehyde (1.95 g, 15.0 mmol) is dissolved in 75 ml methanol/acetic acid (93/7), then 1.65 g (16.5 mmol) 4-methyl-piperazine followed by 1.20 g (15.0 mmol) sodium cyanoborohydride are added. This mixture is stirred at room temperature for 20 hours. Afterwards 5 ml 2N hydrochloric acid is added, and stirring is continued for 20 minutes at room temperature. After addition of 40% NaOH to basify the solution, the title compound is extracted with ethylacetate. The crude product is purified by chromatography on silica (ethylacetate/methanol/ammonia:9/1/0.1).

¹H-NMR (400 MHZ, DMSO-d6): 2.13 (s, 3H), 2.20-2.45 (m, 8H), 3.44 (s, 2H), 4.11 (s, 1H), 7.25 (d, 2H), 7.40 (d, 2H).

MS (ESI⁺) m/z: 215 [MH]⁺

### b) 1-Methyl-4-{4-[(E)-2-(4,4,5,5-tetramethyl-[1,3,2]dioxaborolan-2-yl)-vinyl]-benzyl}-piperazine

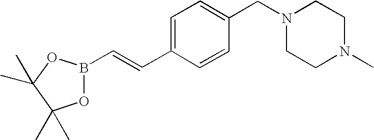

1-(4-Ethynyl-benzyl)-4-methyl-piperazine (0.60 g, 2.8 mmol) is dissolved in 30 ml dichloromethane and 1.07 g (8.4 mmol) 4,4,5,5-tetramethyl-[1,3,2]dioxaborolane is added. The solution is degassed by introduction of a stream of argon, Rh[P(Ph)₃]₃Cl (104 mg, 0.056 mmol) is added and the mixture is stirred at room temperature for 24 hours. The reaction is quenched with saturated ammonium chloride solution, extracted into ethylacetate, the organic phase is dried over Na2SO4 and the solvent evaporated. The crude is purified by chromatography on silica (ethylacetate/methanol/ammonia:7/3/0.3) to yield the desired boronate.

¹H-NMR (400 MHZ, DMSO-d6): 1.23 (s, 12H), 2.16 (s, 3H), 2.25-2.45 (m, 8H), 3.44 (s, 2H), 6.10 (d, 1H), 7.20-7.30 (m, 3H), 7.49 (d, 2H).

MS (ESI⁺) m/z: 343 [MH]⁺

### c) 6-(2-{(E)-2-[4-(4-Methyl-piperazin-1ylmethyl)phenyl]-vinyl}-pyridin-4-yl)-3,7-dihydro-pyrrolo[2,3-d]pyrimidine-one

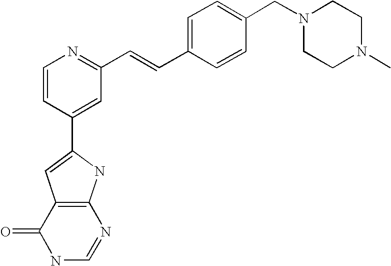

1-Methyl-4-{4-[(E)-2-(4,4,5,5-tetramethyl-[1,3,2]dioxaborolan-2-yl)-vinyl]-benzyl}-piperazine (100 mg, 0.29 mmol) and 6-(2-Chloro-pyridin-4-yl)-3,7-dihydropyrrolo[2,3-d]pyrimidine-4-one (86.4 mg, 0.35 mmol) are dissolved in 3 ml n-propanol and 1 ml 2N sodium carbonate solution. The solution is degassed by introduction of a stream of argon, Pd[P(Ph)₃]₂Cl₂ (20 mg, 0.03 mmol) is added and the mixture is heated to 140° C. for 15 minutes in a microwave oven. After evaporation of the solvents the crude is purified by chromatography on silica (ethylacetate/methanol/ammonia:8/2/0.2 to 7/3/0.3).

¹H-NMR (400 MHZ, DMSO-d6): 2.47 (s, 3H), 2.48-2.2.65 (m, 8H), 3.55 (s, 2H), 7.20-7.35 (m, 5H), 7.61-7.69 (m, 3H), 7.94 (s, 1H), 8.04 (s, 1H), 8.51 (d, 1H), 11.92 (brs 1 NH), 12.59 (brs, 1 NH).

MS (ESI⁺) m/z: 427 [MH]⁺

### EXAMPLE 21

### 6-{2-[(E)-2-(4-Diethylaminomethyl-phenyl)-vinyl]-pyridin-4-yl}-3,7-dihydro-pyrrolo[2,3-d]pyrimidin-4-one

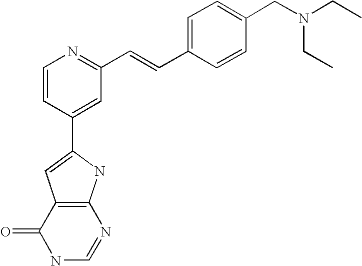

Starting from 6-(2-chloro-pyridin-4-yl)-3,7-dihydropyrrolo[2,3-d]pyrimidine-4-one (187 mg, 0.76 mmol) and diethyl-{4-[(E)-2-(4,4,5,5-tetramethyl-[1,3,2]dioxaborlan-2-yl)-vinyl]-benzyl}-amine (200 mg, 0.63 mmol), the title compound is prepared as described in example 20). After purification by column chromatography on silica gel (ethylacetate/methanol/ammonia:9/1/0.1 to 8/2/0.2), the desired product is obtained.

¹H-NMR (400 MHZ, DMSO-d6): 0.99 (t, 6H), 2.48 (m, 4H), 3.54 (s, 2H), 7.20-7.40 (m, 4H), 7.55-7.72 (m, 4H), 7.92 (s, 1H), 8.03 (s, 1H), 8.51 (d, 1H), 11.92 (brs 1 NH), 12.58 (brs, 1 NH).

MS (ESI⁺) m/z: 400 [MH]⁺

### EXAMPLE 22

### 2,2-Dimethyl-propionic acid 4-{(E)-2-[4-(4-oxo-4,7-dihydro-3H-pyrrolo[2,3-d]pyrimidin-6yl)-pyridin-2-yl]-vinyl}-benzyl Ester

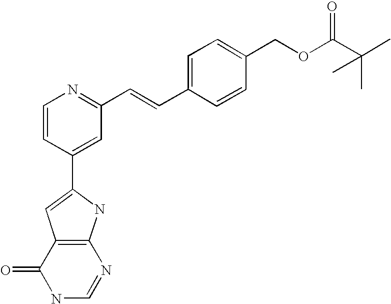

Starting from 6-(2-chloro-pyridin-4-yl)-3,7-dihydropyrrolo[2,3-d]pyrimidine-4-one (483 mg, 1.96 mmol) and 2,2-dimethyl-propionic acid 4-[(E)-2-(4,4,5,5-tetramethyl-[1,3,2]dioxaborolan-2-yl)-vinyl]-benzyl ester (450 mg, 1.31 mmol), the title compound is prepared as described in example 20). After purification by column chromatography on silica gel (ethylacetate/methanol/ammonia:9/1/0.1 to 8/2/0.2), the desired product is, obtained.

¹H-NMR (400 MHZ, DMSO-d6): 1.18 (s, 9H), 5.10 (s, 2H), 7.28-7.39 (m, 4H), 7.64-7.72 (m, 4H), 7.91 (s, 1H), 8.04 (s, 1H), 8.51 (d, 1H), 12.00-12.40 (m, 2NH).

MS (ESI⁺) m/z: 429 [MH]⁺

### EXAMPLE 23

### 2,2-Dimethyl-propionic acid 2-{(E)-2-[4-(4-oxo-4,7-dihydro-3H-pyrrolo[2,3-d]pyrimidin-6yl)-pyridin-2-yl]-vinyl}-benzyl Ester

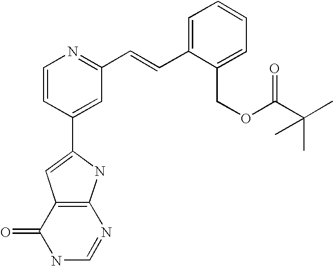

Starting from 6-(2-chloro-pyridin-4-yl)-3,7-dihydropyrrolo[2,3-d]pyrimidine-4-one (429 mg, 1.75 mmol) and 2,2-dimethyl-propionic acid 2-[(E)-2-(4,4,5,5-tetramethyl-[1,3,2]dioxaborolan-2-yl)-vinyl]-benzyl ester (400 mg, 1.16 mmol), the title compound is prepared as described in example 20). After purification by column chromatography on silica gel (ethylacetate/methanol/ammonia:9/1/0.1 to 8/2/0.2), the desired product is obtained.

¹H-NMR (400 MHZ, DMSO-d6): 1.12 (s, 9H), 5.30 (s, 2H), 7.17 (d, 1H), 7.29 (s, 1H), 7.35-7.45 (m, 3H), 7.68 (d, 1H), 7.81 (d, 1H), 7.88-7.95 (m, 2H), 8.04 (s, 1H), 8.54 (d, 1H), 11.90 (brs, 1 NH), 12.62 (brs, 1NH).

MS (ESI⁺) m/z: 429 [MH]⁺

### EXAMPLE 24

### 6-{2-[(E)-2-(4-Piperidin-1-ylmethyl-phenyl)-vinyl]-pyridin-4-yl}-3,7-dihydro-pyrrolo[2,3-d]pyrimidin-4-one

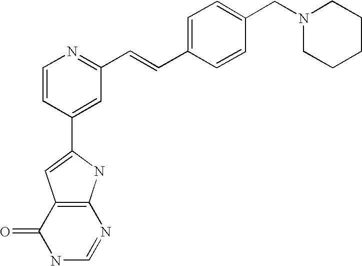

Starting from 6-(2-chloro-pyridin-4-yl)-3,7-dihydropyrrolo[2,3-d]pyrimidine-4-one (128 mg, 0.52 mmol) and 1-{4-[(E)-2-(4,4,5,5-tetramethyl-[1,3,2]dioxaborolan-2-yl)-vinyl]-benzyl}-piperidine (170 mg, 0.52 mmol), the title compound is prepared as described in example 20). After purification by column chromatography on silica gel (ethylacetate/methanol/ammonia:9/1/0.1 to 8/2/0.2), the desired product is obtained.

¹H-NMR (400 MHZ, DMSO-d6): 1.39 (m, 2H), 1.51 (m, 4H), 2.33 (m, 4H), 3.44 (s, 2H), 7.20-7.33 (m, 4H), 7.59 (d, 1H), 7.65-7.73 (m, 2H), 7.80 (d, 1H), 7.94 (s, 1H), 8.05 (s, 1H), 8.53 (m, 1H), 11.93 (brs, 1NH), 12.59 (brs, 1NH).

MS (ESI⁺) m/z: 412 [MH]⁺

### EXAMPLE 25

### 4-(4-{(E)-2-[4-(4-Oxo-4,7-dihydro-3H-pyrrolo[2,3-d]pyrimidin-6-yl)-pyridin-2-yl]-vinyl}-benzyl)-piperazine-1-carboxylic acid tert.-butyl Ester

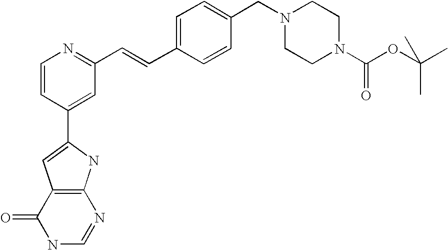

Starting from 6-(2-chloro-pyridin-4-yl)-3,7-dihydropyrrolo[2,3-d]pyrimidine-4-one (172 mg, 0.70 mmol) and 4-{4-[(E)-2-(4,4,5,5-tetramethyl-[1,3,2]dioxaborolan-2-yl)-vinyl]benzyl}-piperazine-1-carboxylic acid tert.-butyl ester (250 mg, 0.58 mmol), the title compound is prepared as described in example 20). After purification by column chromatography on silica gel (ethylacetate/methanol/ammonia:8/2/0.2), the desired product is obtained.

¹H-NMR (400 MHZ, DMSO-d6): 1.43 (s, 9H), 2.30-2.40 (m, 4H), 3.25-3.30 (m, 4H), 3.45-3.55 (m, 2H), 7.20-7.40 (m, 4H), 7.55-7.75 (m, 4H), 7.81 (d, 1H), 7.93 (s, 1H), 8.04 (s, 1H), 11.90 (brs 1 NH), 12.55 (brs, 1 NH).

MS (ESI⁺) m/z: 513 [MH]⁺

### EXAMPLE 26

### 6-{2-[(E)-2-(3-Fluoro-4-morpholin-4-ylmethyl-phenyl)-vinyl]-pyridin-4-yl}-3,7-dihydro-pyrrolo[2,3-d]pyrimidin-4-one

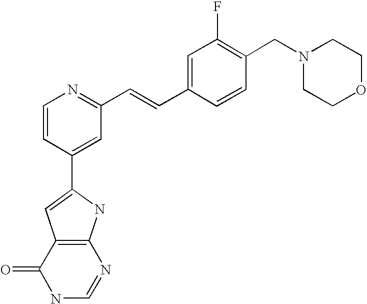

Starting from 6-(2-chloro-pyridin-4-yl)-3,7-dihydropyrrolo[2,3-d]pyrimidine-4-one (511 mg, 2.07 mmol) and 4-{2-fluoro-4-[(E)-2-(4,4,5,5-tetramethyl-[1,3,2]dioxaborolan-2-yl)-vinyl]-benzyl}-morpholine (600 mg, 1.72 mmol), the title compound is prepared as described in example 20). After purification by column chromatography on silica gel (ethylacetate/methanol/ammonia:9/1/0.1 to 85/15/1.5), the desired product is obtained.

¹H-NMR (400 MHZ, DMSO-d6): 2.39 (brs 4H), 3.52 (s, 2H), 3.57 (brs, 4H), 7.25-7.30 (m, 2H), 7.35-7.50 (m, 3H), 7.70-7.75 (m, 2H), 7.93 (d, 1H), 8.03 (s, 1H), 8.53 (d, 1H), 11.93 (brs, 1NH), 12.60 (brs, 1NH).

MS (ESI⁺) m/z: 432 [MH]⁺

### EXAMPLE 27

### 6-{2-[-2-(3-Fluoro-4-morpholinylmethyl-phenyl)-ethyl]-pyridin-4-yl}-3,7-dihydro-pyrrolo[2,3-d]pyrimidin-4-one

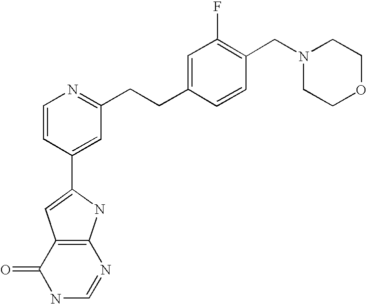

6-{2-[(E)-2-(3-Fluoro-4-morpholin-4-ylmethyl-phenyl)-vinyl]-pyridin-4-yl}-3,7-dihydro-pyrrolo[2,3-d]pyrimidin-4-one (example 26) (160 mg 0.37 mmol) is dissolved in 10 ml methanol and 10 dichloromethane. The solution is hydrogenated at room temperature and atmospheric pressure over night in the presence of 40 mg palladium (10% on activated charcoal). The mixture is filtered and evaporated. The solid formed after evaporation is triturated with diethyl ether to give the target molecule.

¹H-NMR (400 MHZ, DMSO-d6): 2.77 (brs, 4H), 3.10-3.15 (m, 2H), 3.70-3.73 (m, 4H), 3.88 (s, 2H), 7.05-7.15 (m, 3H), 7.46 (t, 1H), 7.65 (d, 1H), 7.72 (s, 1H), 7.83 (s, 1H), 8.46 (d, 1H), 11.44 (brs, 1NH), 12.22 (brs, 1NH).

MS (ESI⁺) m/z: 434 [MH]⁺

### EXAMPLE 28

### 6-{2-[(E)-2-(3-Morpholin-4-ylmethyl-phenyl)-vinyl]-pyridin-4-yl}-3,7-dihydro-pyrrolo[2,3-d]pyrimidin-4-one

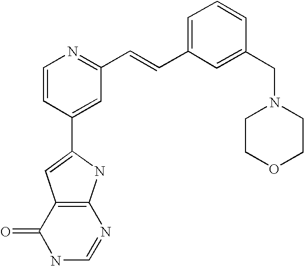

Starting from 6-(2-chloro-pyridin-4-yl)-3,7-dihydropyrrolo[2,3-d]pyrimidine-4-one (112 mg, 0.46 mmol) and 4-{3-[(E)-2-(4,4,5,5-tetramethyl-[1,3,2]dioxaborolan-2-yl]-benzyl}-morpholine (100 mg, 0.30 mmol), the title compound is prepared as described in example 20). After purification by column chromatography on silica gel (ethylacetate/methanol/ammonia:9/1/0.1), the desired product is obtained.

¹H-NMR (400 MHZ, DMSO-d6): 2.39 (brs, 4H), 3.51 (s, 2H), 3.59 (brs, 4H), 7.20-7.39 (, 4H), 7.52-7.60 (m, 2H), 7.65-7.75 (m, 2H), 7.93 (s, 1H), 8.09 (s, 1H), 8.54 (d, 1H), 11.93 brs, 1 NH), 12.57 (brs, 1 NH).

MS (ESI⁺) m/z: 414 [MH]⁺

### EXAMPLE 29

### 6-(2-{(E)-2-[4-(2-Morpholin-4-yl-2-oxo-ethyl)-phenyl]-vinyl}-pyridin-4-yl)-3,7-dihydro-pyrrolo[2,3-d]pyrimidin-4-one

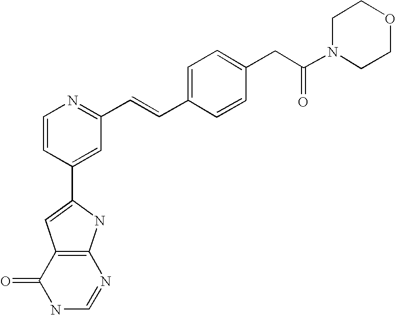

Starting from 6-(2-Chloro-pyridin-4-yl)-3,7-dihydropyrrolo[2,3-d]pyrimidine-4-one (143 mg, 0.58 mmol) and 1-Morpholin-4-yl-2-{4-[(E)-2-(4,4,5,5-tetramethyl[1,3,2]dioxaborolan-2-yl)-vinyl]-phenyl}-ethanone (250 mg, 0.70 mmol), the title compound is prepared as described in example 20). After purification by column chromatography on silica gel (ethylacetate/methanol/ammonia:9/1/0.1 to 8/2/0.2), the desired product is obtained.

¹H-NMR (400 MHZ, DMSO-d6): 3.45-3.55 (m, 8H), 3.75 (s, 2H), 7.18-7.25 (m, 3H), 7.59 (d, 2H), 7.65-7.75 (m, 2H), 7.80 (d, 1H), 7.93 (s, 1H), 8.05 (s, 1H), 8.54 (m, 1H), 11.95 brs, 1 NH), 12.59 (brs, 1 NH).

MS (ESI⁺) m/z: 442 [MH]⁺

### EXAMPLE 30

### 6-[2-(4-Morpholin-4-ylmethyl-phenylethynyl)-pyridin-4-yl]-3,7-dihydro-pyrrolo[2,3-d]pyrimidin-4-one

### a) 4-(4-Ethynyl-benzyl)-morpholine

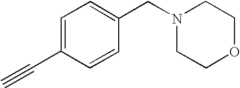

4-Ethynylbenzaldehyde (1.30 g, 10.0 mmol) are dissolved in 50 ml methanol/acetic acid (93/7), then 0.96 g (11.0 mmol) morpholine followed by 0.80 g (10.0 mmol) sodium cyanoborohydride are added. This mixture is stirred at room temperature for 20 hours. 5 ml 2N hydrochloric acid is added and stirring is continued for 20 minutes. After addition of 40% NaOH to basify the solution, the title compound is extracted with ethylacetate. The crude is purified by chromatography on silica gel (ethylacetate/methanol/ammonia:9/1/0.1).

¹H-NMR (400 MHZ, DMSO-d6): 2.32 (t, 4H), 3.45 (s, 2H), 3.55 (t, 4H), 4.12 (s, 1H), 7.29 (d, 2H), 7.40 (d, 2H).

MS (ESI⁺) m/z: 202 [MH]⁺

### b) 6-[2-(4-Morpholin-4-ylmethyl-phenylethynyl)-pyridin-4-yl]-3,7-dihydro-pyrrolo[2,3-d]pyrimidin-4-one

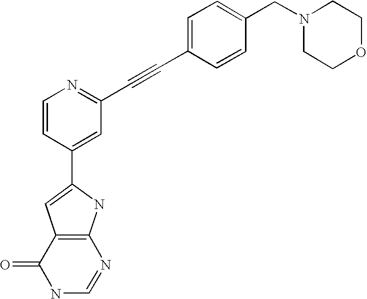

4-(4-Ethynyl-benzyl)morpholine (200 mg, 1.0 mmol) and 6-(2-chloro-pyridin-4-yl)-3,7-dihydropyrrolo[2,3-d]pyrimidine-4-one (122 mg, 0.50 mmol) are dissolved in 5 ml n-propanol and 1 ml 2N sodium carbonate solution. The solution is degassed by introduction of a stream of argon, Pd[P(Ph)₃]₂Cl₂ (30 mg, 0.05 mmol) is added and the mixture is heated to 160° C. for 15 minutes in a microwave oven. After evaporation of the solvents the crude is purified by chromatography on silica (ethylacetate/methanol/ammonia:9/1/0.1).

¹H-NMR (400 MHZ, DMSO-d6): 2.30-2.50 (m, 4H), 3.30 (s, 2H), 3.35-3.55 (m, 4H), 7.39 (m, 3H), 7.57 (d, 2H), 7.81 (d, 1H), 7.93 (s, 1H), 8.11 (s, 1H), 8.55 (d, 1H), 11.95 (brs, 1NH), 12.60 (brs, 1NH).

MS (ESI⁺) m/z: 412 [MH]⁺

### EXAMPLE 31

### 6-{2-[2-(4-Morpholin-4-ylmethyl-phenyl)-ethyl]-pyridin-4-yl}-3,7-dihydro-pyrrolo[2,3-d]pyrimidine-one

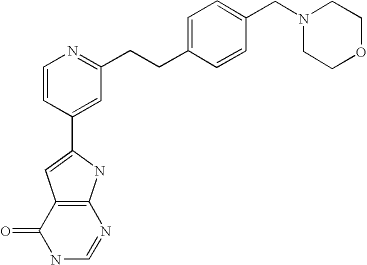

6-[2-(4-Morpholin-4-ylmethyl-phenylethynyl)-pyridin-4-yl]-3,7-dihydro-pyrrolo[2,3-d]pyrimidin-4-one (150 mg 0.36 mmol) (example 30) is dissolved in 20 ml ethanol. The solution is hydrogenated at room temperature and atmospheric pressure over night in the presence of 50 mg palladium 10% on activated charcoal. The mixture is filtrated and evaporated. The solid formed after evaporation is titurated with diethyl ether to give the target molecule.

¹H-NMR (400 MHZ, DMSO-d6): 2.29 (m, 4H), 3.01 (m, 2H), 3.38 (s, 2H), 3.51 (m, 4H), 7.15-7.20 (m, 5H), 7.60 (d, 1H), 7.67 (s, 1H), 7.90 (s, 1H), 8.43 (d, 1H), 11.89 (brs, 1NH), 12.50 (brs 1NH).

MS (ESI⁺) m/z: 416 [MH]⁺

### EXAMPLE 32

### 6-{2-[(E)-2-(4-Morpholin-4-ylmethyl-phenyl)-vinyl]-pyridin-4-yl}-3,7-dihydro-pyrrolo[2,3-d]pyrimidin-4-one

### a) 4-{4-[(E)-2-(4,4,5,5-Tetramethyl-[1,3,2]dioxaborolan-2-yl)-vinyl]-benzyl}-morpholine

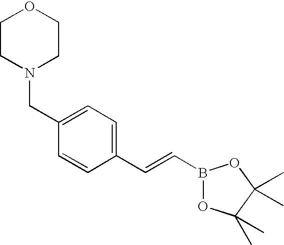

Hydroboration of 4-(4-ethynyl-benzyl)-morpholine (example 30) (1.5 g, 7.45 mmol) as described in example 20) and purification by chromatography on silica (ethylacetate/hexanes) yields the desired vinyl-boronate

¹H-NMR (400 MHZ, DMSO-d6): 1.24 (s, 12H), 2.45 (br t, 4H), 3.49 (s, 2H), 2.71 (t, 4H), 6.16 (d, 1H), 7.30 (d, 2H), 7.39 (d, 1H), 7.44 (d, 2H).

MS (ESI⁺) m/z: 330 [MH]⁺

### b) 6-{2-[(E)-2-(4-Morpholin-4-ylmethyl-phenyl)-vinyl]-pyridin-4-yl}-3,7-dihydro-pyrrolo[2,3-d]pyrimidin-4-one

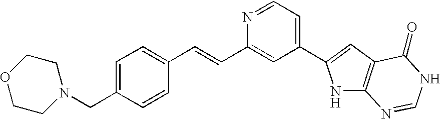

Coupling of 6-(2-Chloro-pyridin-4-yl)-3,7-dihydro-pyrrolo[2,3-d]pyrimidin-4-one (500 mg, 1.77 mmol) with 4-{4-[(E)-2-(4,4,5,5-tetramethyl-[1,3,2]dioxaborolan-2-yl)-vinyl]-benzyl}-morpholine (870 mg, 2.65 mmol) as described in Example 1d) gives the title compound.

¹H-NMR (400 MHZ, DMSO-d6): 2.36 (brs, 4H), 3.47 (s, 2H), 3.57 (brs, 4H), 7.22 (d, 1), 7.29 (s, 1H), 7.34 (d, 2H), 7.60 (d, 2H), 7.65 (d, 1H), 7.70 (d, 1H), 7.92 (s, 1H), 8.04 (s, 1H), 8.51 (d, 1H), 11.9 (brs, 1H), 12.5 (brs, 1H).

MS (ESI⁺) m/z: 414 [MH]⁺

### EXAMPLE 33

### N,N-Diethyl-4-{(E)-2-[4-(4-oxo-4,7-dihydro-3H-pyrrolo[2,3-d]pyrimidin-6-yl)-pyridin-2-yl]-vinyl}-benzamide

### a) N,N-Diethyl-4-ethynyl-benzamide

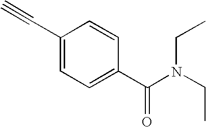

4-Ethynylbenzoic acid sodium salt (1.0 g, 5.77 mmol), HOBT (1.0 g, 6.51 mmol) and diethylamine (1.2 ml, 11 mmol) are suspended in 50 ml CH₂Cl₂/THF (1:1), then EDC hydrochloride (1.3 g, 6.78 mmol) is added at r.t. The resulting clear reaction mixture is stirred over night, quenched with saturated aqueous NaHCO₃ solution and extracted with ethylacetate. The organic layer is washed with water and brine, dried over Na₂SO₄ and concentrated in vacuo. Silica gel purification (hexanes/ethylacetate) affords the product as a colorless solid.

¹H-NMR (400 MHZ, CDCl₃): 1.00-1.10 (m, 3H), 1.15-1.25 (m, 3H), 3.11 (s, 1H), 3.15-3.25 (m, 2H), 3.47-3.57 (m, 2H), 7.31 (d, 2H), 7.49 (d, 2H).

MS (ESI⁺) m/z: 202 [MH]⁺

### b) N,N-Diethyl-4-[(E)-2-(4,4,5,5-tetramethyl-[1,3,2]dioxaborolan-2-yl)-vinyl]-benzamide

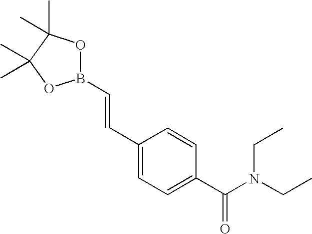

Under Ar N,N-Diethyl-4-ethynyl-benzamide (900 mg, 4.34 mmol) and Wilkinson's catalyst (RhCl(PPh₃)₃) (85 mg, 0.08 mmol) are dissolved in CH₂Cl₂. A solution of pinacolborane (1.2 g, 9.2 mmol) in 3 ml CH₂Cl₂ is slowly added and the resulting dark red reaction mixture is allowed to stir at r.t. for 24 h. The reaction is quenched with ice-water and extracted with ethylacetate. The organic layer is washed with water and brine, dried over Na₂SO₄ and concentrated in vacuo. After filtration over silica gel (hexanes/ethylacetate) the product is used in the next step without further purification.

MS (ESI⁺) m/z: 330 [MH]⁺

### c) N,N-Diethyl-4-{(E)-2-[4-(4-oxo-4,7-dihydro-3H-pyrrolo[2,3-d]pyrimidin-6-yl)-pyridin-2-yl]-vinyl}-benzamide

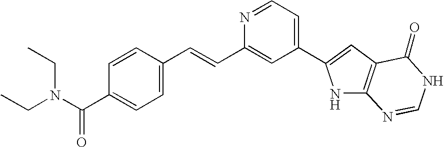

N,N-Diethyl-4-[(E)-2-(4,4,5,5-tetramethyl-[1,3,2]dioxaborolan-2-yl)-vinyl]-benzamide (145 mg, 0.43 mmol), 6-(2-chloro-pyridin-4-yl)-3,7-dihydro-pyrrolo[2,3-d]pyrimidin-4-one (60 mg, 0.24 mmol), 2 M Na₂CO₃ (0.5 ml, 1.0 mmol) and Pd(PPh₂)₂Cl₂ (5 mg, 0.006 mmol) are suspended in 2 ml n-propanol, purged with argon and subjected to microwave heating (160° C., 15 min). The reaction mixture is diluted with ethylacetate, washed with water and brine, dried over Na₂SO₄ and concentrated in vacuo. The crude material is purified by silica gel chromatography (CH₂Cl₂/methanol) to afford a yellow crystalline product.

¹H-NMR (400 MHZ, DMSO-d6): 1.03-1.17 (m, 6H), 3.15-3.25 (m, 2H), 3.35-3.47 (m, 2H), 7.28 (s, 1H), 7.30 (s, 1H), 7.37 (s, 2H), 7.66 (s, 1H), 7.66 (s, 2H), 7.71 (s, 1H), 7.92 (s, 1H), 8.05 (s, 1H), 8.54 (d, 1H), 11.92 (s, 1H) 12.60 (s, 1H).

MS (ESI⁺) m/z: 414 [MH]⁺

The following compounds are prepared in analogous manner:

### EXAMPLE 34

### 6-(2-{(E)-2-[4-(Morpholine-4-carbonyl)-phenyl]-vinyl}-pyridin-4-yl)-3,7-dihydro-pyrrolo[2,3-d]pyrimidin-4-one

### a) (4-Ethynyl-phenyl)-morpholin-4-yl-methanone

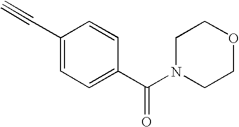

¹H-NMR (400 MHZ, DMSO-d6): 3.45-3.65 (m, 8H), 4.31 (s, 1H), 7.41 (d, 2H), 7.52 (d, 2H).

MS (ESI⁺) m/z: 216 [MH]⁺

### b) Morpholin-4-yl-{4-[(E)-2-(4,4,5,5-tetramethyl-[1,3,2]dioxaborolan-2-yl)-vinyl]-phenyl}-methanone

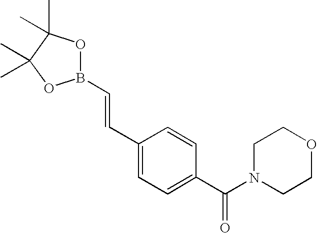

MS (ESI⁺) m/z: 344 [MH]⁺

### c) 6-(2-{(E)-2-[4-(Morpholine-4-carbonyl)-phenyl]-vinyl}-pyridin-4-yl)-3,7-dihydro-pyrrolo[2,3-d]pyrimidin-4-one

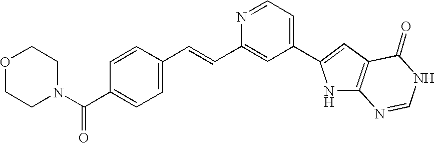

¹H-NMR (400 MHZ, DMSO-d6): 3.55-3.65 (m, 8), 7.34 (s, 1H), 7.39 (s, 1H), 7.44 (d, 2H), 7.68 (d, 1H), 7.71 (d, 2H), 7.76 (s, 1H), 7.92 (s, 1H), 8.07 (s, 1H), 8.54 (d, 1H), 11.93 (s, 1H) 12.61 (s, 1H).

MS (ESI⁺) m/z: 428 [MH]⁺

### EXAMPLE 35

### 6-(2-{(E)-2-[4-(4-Hydroxy-piperidine-1-carbonyl)-phenyl]-vinyl}-pyridin-4-yl)-3,7-dihydro-pyrrolo[2,3-d]pyrimidin-4-one

### a) (4-Ethynyl-phenyl)-(4-hydroxy-piperidin-1-yl)-methanone

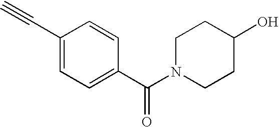

¹H-NMR (400 MHZ, CDCl₃): 1.40-2.05 (m, 4H), 1.70 (s, 1H), 3.13 (s, 1H), 3.10-4.25 (m, 5H), 7.34 (d, 2H), 7.51 (d, 2H).

MS (ESI⁺) m/z: 230 [MH]⁺

### b) (4-Hydroxy-piperidin-1-yl)-{4-[(E)-2-(4,4,5,5-tetramethyl-[1,3,2]dioxaborolan-2-yl)-vinyl]-phenyl}-methanone

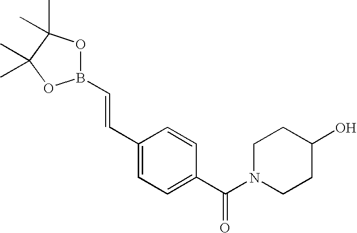

### c) 6-(2-{(E)-2-[4-(4-Hydroxy-piperidine-1-carbonyl)-phenyl]-vinyl}-pyridin-4-yl)-3,7-dihydro-pyrrolo[2,3-d]pyrimidin-4-one

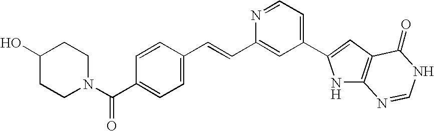

¹H-NMR (400 MHZ, DMSO-d6): 1.30-1.40 (m, 2H), 1.65-1.85 (m, 2H), 3.10-3.25 (m, 3H), 3.45-3.60 (m, 1H), 3.70-3.78 (m, 1H), 4.81 (s, 1H), 7.30 (s, 1H), 7.33 (s, 1H), 7.40 (d, 2H), 7.68 (s, 1H), 7.70 (d, 2H), 7.77 (s, 1H), 7.92 (s, 1H), 8.09 (s, 1H), 8.54 (d, 1H), 11.96 (s, 1H) 12.62 (s, 1H).

MS (ESI⁺) m/z: 442 [MH]⁺

### EXAMPLE 36

### 6-(2-{(E)-2-[3-(Morpholine-4-carbonyl)-phenyl]-vinyl}-pyridin-4-yl)-3,7-dihydro-pyrrolo[2,3-d]pyrimidin-4-one

### a) (3-Ethynyl-phenyl)-morpholin-4-yl-methanone

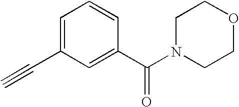

¹H-NMR (400 MHZ, DMSO-d6): 3.20-3.70 (m, 8H), 4.27 (s, 1H), 7.40-7.50 (m, 3H), 7.53-7.57 (m, 1H).

MS (ESI⁺) m/z: 216 [MH]⁺

### b) Morpholin-4-yl-{3-[(E)-2-(4,4,5,5-tetramethyl-[1,3,2]dioxaborolan-2-yl)-vinyl]-phenyl}-methanone

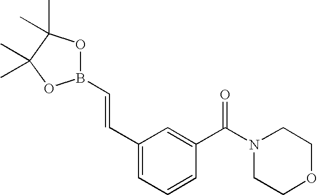

MS (ESI⁺) m/z: 344 [MH]⁺

### c) 6-(2-{(E)-2-[3-Morpholine-4-carbonyl)-phenyl]-vinyl}-pyridin-4-yl)-3,7-dihydro-pyrrolo[2,3-d]pyrimidin-4-one

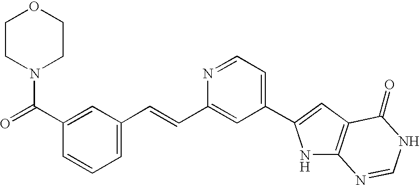

¹H-NMR (400 MHZ, DMSO-d6): 3.53-3.72 (m, 8H), 7.24-7.49 (m, 4H), 7.66-7.81 (m, 4H), 7.92 (s, 1H), 8.12 (s, 1H), 8.52 (d, 1H), 12.00 (s, 1H) 12.71 (s, 1H).

MS (ESI⁺) m/z: 428 [MH]⁺

### EXAMPLE 37

### 6-[2-((E)-2-Pyridin-3-yl-vinyl)-pyridin-4-yl]-3,7-dihydro-pyrrolo[2,3-d]pyrimidin-4-one

### a) 3-[(E)-2-(4,4,5,5-Tetramethyl-[1,3,2]dioxaborolan-2-yl)-vinyl]-pyridine

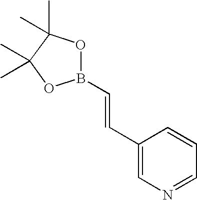

The title compound is prepared as described in Example 32b) starting from 3-ethylnylpyridine (0.95 g, 9.03 mmol) and pinacolborane (2.40 g, 18.2 mmol). Colorless crystals are obtained, which are used directly in the next step.

### b) 2-Amino-5-[2-((E)-2-pyridin-3-yl-vinyl)-pyridin-4-yl]-1H-pyrrole-3-carboxylic acid Ethyl Ester

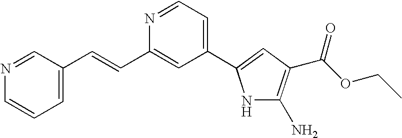

2-Amino-5-(2-chloro-pyridin-4-yl)-1H-pyrrole-3-carboxylic acid ethyl ester (85 mg, 0.32 mmol), 3-[(E)-2-(4,4,5,5-tetramethyl-[1,3,2]dioxaborolan-2-yl)-vinyl]-pyridine (140 mg, 0.61 mmol), 2 M Na₂CO₃ (0.1 ml, 2.0 mmol) and Pd(PPh₂)₂Cl₂ (20 mg, 0.028 mmol) are suspended in 4 ml n-propanol, purged with argon and subjected to microwave heating (160° C., 15 min). The reaction mixture is loaded on Isolute® Sorbent and purified by silica gel chromatography (ethylacetate/methanol) to afford a yellow solid.

MS (ESI⁺) m/z: 335 [MH]⁺

### c) 6-[2-((E)-2-Pyridin-3-yl-vinyl)-pyridin-4-yl]-3,7-dihydro-pyrrolo[2,3-d]pyrimidin-4-one

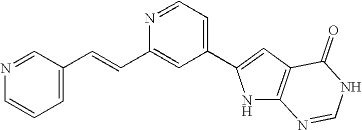

The title compound is prepared as described in Example 1b) starting from 2-amino-5-[2-((E)-2-pyridin-3-yl-vinyl)-pyridin-4-yl]-1H-pyrrole-3-carboxylic acid ethyl ester (135 mg, 0.40 mmol) and formamidine hydrochloride (170 mg, 2.11 mmol). Red crystals are obtained.

¹H-NMR (400 MHZ, DMSO-d6): 7.29 (s, 1H), 7.38 (d, 1H), 7.40-7.45 (m, 1H), 7.70 (d, 1H), 7.75 (d, 1H), 7.92 (s, 1H), 8.10 (d, 1H), 8.12 (s, 1H), 8.49 (d, 1H), 8.83 (d, 1H), 8.80 (d, 1H), 12.38 (brs, 2H).

MS (ESI⁺) m/z: 316 [MH]⁺

### EXAMPLE 38

### 6-(2-{(E)-2-[4-(4-Acetyl-piperazin-1-ylmethyl)-phenyl]-vinyl}-pyridin-4-yl)-3,7-dihydro-pyrrolo[2,3-d]pyrimidin-4-one

### a) 1-[4-(4-Ethynyl-benzyl)piperazin-1-yl]-ethanone

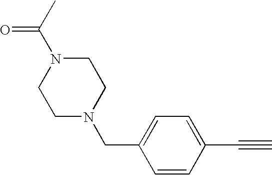

¹H-NMR (400 MHZ, DMSO-d6): 1.97 (s, 3H), 2.29 (t, 2H), 2.36 (t, 2H), 3.38-3.45 (m, 4H), 3.49 (s, 2H), 4.15 (s, 1H), 7.30 (d, 2H), 7.43 (d, 2H).

MS (ESI⁺) m/z: 243 [MH]⁺

### b) 1-(4-{4-[(E)-2-(4,4,5,5-Tetramethyl-[1,3,2]dioxaborolan-2-yl)-vinyl]-benzyl}-piperazin-1-yl)ethanone

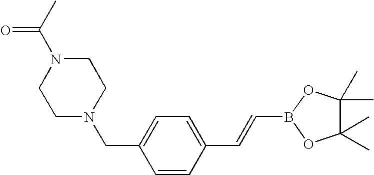

MS (ESI⁺) m/z: 371 [MH]⁺

### c) 6-(2-{(E)-2-[4-(4-Acetyl-piperazin-1-ylmethyl)-phenyl]-vinyl}-pyridin-4-yl)-3,7-dihydro-pyrrolo[2,3-d]pyrimidin-4-one

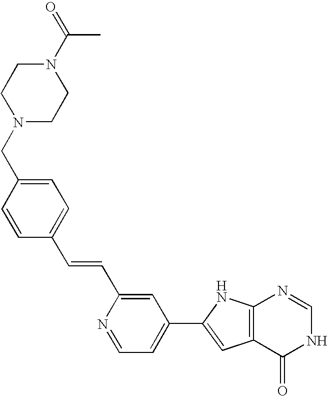

¹H-NMR (400 MHZ, DMSO-d6): 1.99 (s, 3H), 2.28-2.44 (m, 4H). 3.38-3.48 (m, 4H), 3.53 (s, 2H), 7.24 (d, 2H), 7.32 (s, 1H), 7.36 (d, 2H), 7.63 (d, 2H), 7.68 (d, 1H), 7.72 (d, 1H), 7.94 (s, 1H), 8.06 (s, 1H), 8.54 (d, 1H), 11.9 (s, 1H), 12.6 (s, 1H).

MS (ESI⁺) m/z: 455 [MH]⁺

### EXAMPLE 39

### 2-Amino-6-{2-[(E)-2-(4-morpholin-4-ylmethyl-phenyl)-vinyl]-pyridin-4-yl}-3,7-dihydro-pyrrolo[2,3-d]pyrimidin-4-one

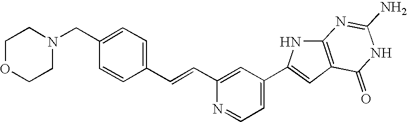

MS (ESI⁺) m/z: 429 [MH]⁺

### EXAMPLE 40

### 6-{2-[(E)-2-(4-Morpholin-4-ylmethyl-phenyl)-vinyl]-pyridin-4-yl}-2-trifluoromethyl-3,7-dihydro-pyrrolo[2,3-d]pyrimidin-4-one

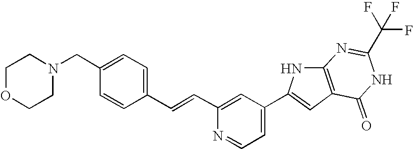

¹H-NMR (400 MHZ, DMSO-d6): 2.36 (br s, 4H), 3.47 (s, 2H), 3.57 (t, 4H), 7.26 (d, 1H), 7.36 (d, 2H), 7.42 (s, 1H), 7.62 (d, 2H), 7.72 (d, 1H), 7.74 (d, 1H), 8.10 (s, 1H), 8.58 (d, 1H), 13.1 (s, 1H), 13.3 (br s, 1H).

MS (ESI⁺) m/z: 482 [MH]⁺

### EXAMPLE 41

### 2-Methyl-6-{2-[(E)-2-(4-morpholin-4-ylmethyl-phenyl)-vinyl]-pyridin-4-yl}-3,7-dihydro-pyrrolo[2,3-d]pyrimidin-4-one

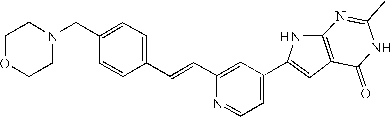

¹H-NMR (400 MHZ, DMSO-d6): 2.30-2.42 (m, 7H), 3.49 (s, 2H), 3.58 (t, 4H), 7.21 (d, 1H), 7.24 (s, 1H), 7.36 (d, 2H), 7.57-7.65 (m, 3H), 7.70 (d, 1H), 8.00 (s, 1H), 8.52 (d, 1H), 11.9 (s, 1H), 12.5 (s, 1H).

MS (ESI⁺) m/z: 428 [MH]⁺

### EXAMPLE 42

### 6-{2-[(E)-2-(4-Hydroxyphenyl)-vinyl]-pyridin-4-yl}-3,7-dihydro-pyrrolo[2,3-d]pyrimidin-4-one

### a) 4-((E)-2-Iodovinyl)-phenol

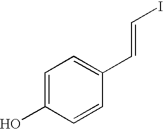

A solution of p-hydroxybenzaldehyde (1.2 g, 10 mmol) and iodoform (7.9 g, 20 mmol) in dry THF (50 ml) is added to a suspension of CrCl₂ (7.4 g, 60 mmol) in THF (50 ml) at 0° C. and the mixture is stirred for 2 h at 0° C. The reaction is quenched by addition of water and mixture is diluted with ethyl acetate. The separated organic layer is washed with saturated Na₂S₂O₃ (aq.) solution, water and brine. It is then dried over Na₂SO₄ and the solvent is evaporated. The product is obtained by further purification of the crude product by flash chromatography.

¹H-NMR (400 MHZ, CDCl₃): 5.16 (s, 1H), 6.62 (d, 1H), 6.81 (d, 2H), 7.20 (d, 2H), 7.34 (d, 1H).

MS (ESI⁻) m/z: 245 [M−H]⁻

### b) 4-[(E)-2-(4,4,5,5-tetramethyl-[1,3,2]dioxaborolan-2-yl)-vinyl]-phenol

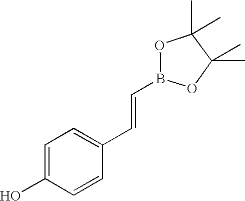

n-BuLi (3.1 ml, 1.6M sol. In hexanes,) is added to a solution of 4-((E)-2-iodovinyl)-phenol (492 mg, 2 mmol) in dry THF (15 ml) at −78° C. Then a solution of 2-isopropyloxy-4,4,5,5-tetramethyl-1,3,2-dioxaborolane (1 ml, 5 mmol) in dry THF (5 ml) is added to the mixture at −78° C. and the whole mixture is stirred at −78° C. for 10 min. followed by stirring at 23° C. for 2 h. The reaction is quenched by addition of saturated NH₄Cl (aq.) solution. After extraction of the mixture with ethyl acetate, the organic layers are washed with brine, dried over Na₂SO₄ and concentrated. The title compound is obtained after flash chromatography.

¹H-NMR (400 MHZ, CDCl₃): 1.34 (s, 12H), 5.06 (s, 1H), 6.03 (d, 1H), 6.82 (d, 2H), 7.31 (d, 1H), 7.41 (d, 2H).

MS (ESI⁻) m/z: 245 [M−H⁺]⁻

### c) 6-{2-[(E)-2-(4-Hydroxyphenyl)-vinyl]-pyridin-4-yl}-3,7-dihydro-pyrrolo[2,3-d]pyrimidin-4-one

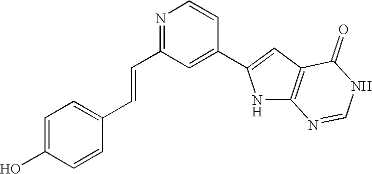

6-(2-Chloro-pyridin-4-yl)-3,7-dihydro-pyrrolo[2,3-d]pyrimidin-4-one (113 mg, 0.4 mmol) and 4-[(E)-2-(4,4,5,5-tetramethyl-[1,3,2]dioxaborolan-2-yl)-vinyl]-phenol (118 mg, 0.48 mmol) are dissolved in n-propanol (3 ml). 1M Na₂CO₃ (aq.) solution (1.6 ml) is added and the mixture is degassed by introduction of a stream of argon. Pd(PPh₂)₂Cl₂ (14 mg, 0.02 mmol) is added and the mixture is irradiated in a microwave oven (SmithCreator, personal chemistry) at 170° C. for 15 min. The reaction is quenched with water, extracted with ethyl acetate, dried (Na₂SO₄) and concentrated on a rotavap. The title product is obtained after flash chromatography of the residue.

¹H-NMR (400 MHZ, DMSO-d6): 6.80 (d, 2H), 7.04 (d, 1H), 7.27 (s, 1H), 7.46 (d, 2H), 7.59 (d, 1H), 7.63 (d, 1H), 7.91 (s, 1H), 7.97 (s, 1H), 8.47 (d, 1H), 9.68 (bs, 1H), 11.94 (bs, 1H), 12.57 (bs, 1H).

MS (ESI⁺) m/z: 331 [MH]⁺

### EXAMPLE 43

### 6-[2-((E)-2-Cyclohexyl-vinyl)-pyridin-4-yl]-3,7-dihydro-pyrrolo[2,3-d]pyrimidin-4-one

### a) ((E)-2-Iodovinyl)-cyclohexane

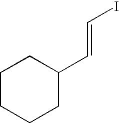

The title product (containing approx. 12% of cis-isomer) is obtained as described in example 42) starting from cyclohexane carboxaldehyde (561 mg, 5 mmol).

¹H-NMR (400 MHZ, CDCl₃): 1.20 (m, 5H), 1.72 (m, 5H), 2.03 (m, 1H), 5.98 (d, 1H), 6.53 (dd, 1H).

MS (ESI⁻) m/z: 239 [M+H₂+H]⁺

### b) 2-((E)-2-Cyclohexyl-vinyl)-4,4,5,5-tetramethyl-[1,3,2]dioxaborolane

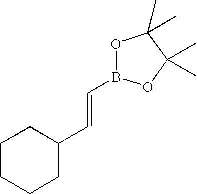

t-BuLi (3.5 ml, 1.7M sol. in pentane, 6 mmol) is added drop wise to a solution of ((E)-2-iodovinyl)-cyclohexane (639 mg, 2.7 mmol) in dry THF (20 ml) at −78° C. After stirring for 1 h at −78° C. 2-isopropyloxy-4,4,5,5-tetramethyl-1,3,2-dioxaborolane (0.86 ml, 4.1 mmol) is added at −78° C. After stirring at −78° C. for 10 min. the mixture is left to stir at room temperature for 2 h. Reaction is quenched by addition of saturated NH₄Cl (aq.) solution and the water layer is extracted with ethyl acetate. Collected organic layers are washed with water and brine, dried (Na₂SO₄) and concentrated. Chromatographic purification of the residue affords the title product.

¹H-NMR (400 MHZ, CDCl₃): 1.11 (m, 4H), 1.27 (s, 12H), 1.66 (m, 2H), 1.74 (m, 5H), 2.04 (m, 1H), 5.36 (d, 1H), 6.57 (dd, 1H).

MS (ESI⁻) m/z: 254 [M+NH₄]⁺

### c) 6-[2-((E)-2-Cyclohexyl-vinyl)-pyridin-4-yl]-3,7-dihydro-pyrrolo[2,3-d]pyrimidin-4-one

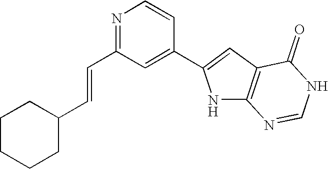

Starting from 6-(2-chloro-pyridin-4-yl)-3,7-dihydro-pyrrolo[2,3-d]pyrimidin-4-one (123 mg, 0.5 mmol) and 2-((E)-2-cyclohexyl-vinyl)-4,4,5,5-tetramethyl-[1,3,2]dioxaborolane (142 mg, 0.6 mmol), the title compound is prepared essentially as described in example 42). After chromatography purification the desired product is obtained.

¹H-NMR (400 MHZ, DMSO-d6): 1.21 (m, 4H), 1.31 (m, 2H), 1.76 (m, 4H), 2.20 (m, 1H), 6.42 (d, 1H), 6.77 (dd, 1H), 7.26 (s, 1H), 7.59 (d, 1H), 7.85 (s, 1H), 7.90 (s, 1H), 8.41 (d, 1H), 11.90 (bs, 1H), 12.52 (bs, 1H).

MS (ESI⁺) m/z: 321 [MH]⁺

### EXAMPLE 44

### 6-(2-{(E)-2-[4-(2-Dimethylamino-ethoxy)-phenyl]-vinyl}-pyridin-4-yl)-3,7-dihydro-pyrrolo[2,3-d]pyrimidin-one

### a) {2-[4-((E)-2-Iodovinyl)-phenoxy]-ethyl}-dimethylamine

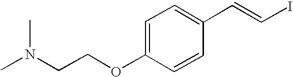

A mixture of 4-((E)-2-iodovinyl)-phenol (443 mg, 1.8 mmol), 2-dimethylaminoethylchloride hydrochloride (389 mg, 2.7 mmol) and K₂CO₃ (746 mg, 5.4 mmol) in acetone (18 ml) is stirred at 50° C. for 2.5 days. After evaporation of the solvent, the mixture is purified by chromatography.

¹H-NMR (400 MHZ, CDCl₃): 2.38 (s, 6H), 2.77 (t, 2H), 4.08 (t, 2H), 6.65 (d, 1H), 6.89 (d, 2H), 7.24 (d, 2H), 7.37 (d, 1H).

MS (ESI⁺) m/z: 318 [MH]⁺

### b) Dimethyl-(2-{4-[(E)-2-(4,4,5,5-tetramethyl-[1,3,2]dioxaborolan-2-yl)-vinyl]-phenoxy}-ethyl)-amine

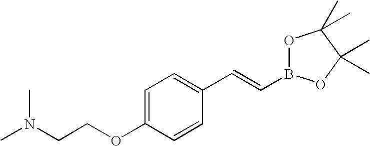

Starting from {2-[4-((E)-2-iodovinyl)-phenoxy]-ethyl}-dimethylamine (327 mg, 1.03 mmol) 130 mg of the title product is obtained as described in example 42b).

¹H-NMR (400 MHZ, CDCl₃): 1.23 (s, 12H), 2.35 (s, 6H), 2.76 (t, 2H), 4.08 (t, 2H), 6.00 (d, 1H), 6.87 (d, 2H), 7.34 (d, 1H), 7.41 (d, 2H).

MS (ESI⁺) m/z: 318 [MH]⁺

### c) 6-(2-{(E)-2-[4-(2-Dimethylamino-ethoxy)-phenyl]-vinyl}-pyridin-4-yl)-3,7-dihydro-pyrrolo[2,3-d]pyrimidin-4-one

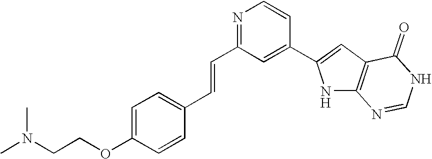

Starting from 6-(2-chloro-pyridin-4-yl)-3,7-dihydro-pyrrolo[2,3-d]pyrimidin-4-one (62 mg, 0.22 mmol) and dimethyl-2-{4-[(E)-2-(4,4,5,5-tetramethyl-[1,3,2]dioxaborolan-2-yl)-vinyl]-phenoxy}-ethyl)-amine (77 mg, 0.24 mmol), the title compound is prepared essentially as described in example 42c). After chromatographic purification the desired product is obtained.

¹H-NMR (400 MHZ, DMSO-d6): 2.21 (s, 6H), 2.62 (t, 2H), 4.07 (t, 2H), 6.98 (d, 2H), 7.11 (d, 1H), 7.16 (s, 1H), 7.56 (d, 2H), 7.58 (d, 1H), 7.60 (d, 1H), 7.80 (s, 1H), 7.95 (s, 1H), 8.42 (d, 1H), 11.96 (bs, 1H).

MS (ESI⁺) m/z: 402 [MH]⁺

### EXAMPLE 45

### 6-(2-{(E)-2-[4-(2-Morpholin-4-yl-ethoxy)-phenyl]-vinyl}-pyridin-4-yl)-3,7-dihydro-pyrrolo[2,3-d]pyrimidin-4-one

### a) 4-{2-[4-((E)-2-Iodovinyl)-phenoxy]-ethyl}-morpholine

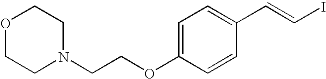

Starting from 4-((E)-2-iodovinyl)phenol (443 mg, 1.8 mmol), 4-(2-chloroethyl)morpholine hydrochloride (502 mg, 2.7 mmol) the title compound is obtained as described in example 44a).

¹H-NMR (400 MHZ, CDCl₃): 2.62 (m, 4H), 2.95 (t, 2H), 3.76 (m, 4H), 4.13 (t, 2H), 6.66 (d, 1H), 6.87 (d, 2H), 7.24 (d, 2H), 7.36 (d, 1H).

MS (ESI⁺) m/z: 360 [MH]⁺

### b) 4-(2-{4-[(E)-2-(4,4,5,5-Tetramethyl-[1,3,2]dioxaborolan-2-yl)-vinyl]-phenoxy}-ethyl)-morpholine

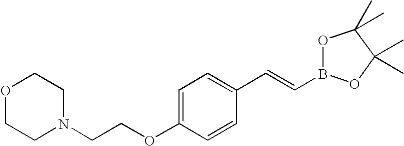

Starting from 4-{2-[4-((E)-2-iodovinyl)phenoxy]-ethyl}-morpholine (539 mg, 1.5 mmol) the title compound is obtained as described in example 42b).

¹H-NMR (400 MHZ, CDCl₃): 1.31 (s, 12H), 2.60 (m, 4H), 2.81 (t, 2H), 3.76 (m, 4H), 4.14 (t, 2H), 6.04 (d, 1H), 6.87 (d, 2H), 7.35 (d, 1H), 7.44 (d, 2H).

MS (ESI⁺) m/z: 360 [MH]⁺

### c) 6-(2-{(E)-2-[4-(2-Morpholin-4-yl-ethoxy)-phenyl]-vinyl}-pyridin-4-yl)-3,7-dihydro-pyrrolo[2,3-d]pyrimidin-4-one

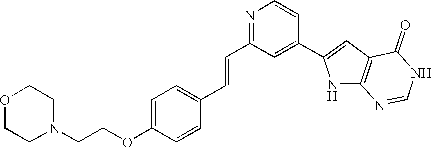

Starting from 6-(2-chloro-pyridin-4-yl)-3,7-dihydro-pyrrolo[2,3-d]pyrimidin-4-one (96 mg, 0.34 mmol) and 4-(2-{4-[(E)-2-(4,4,5,5-tetramethyl-[1,3,2]dioxaborolan-2-yl)-vinyl]-phenoxy}-ethyl)morpholine (147 mg, 0.41 mmol), the title compound is prepared.

¹H-NMR (400 MHZ, DMSO-d6): 2.47 (m, 4H), 2.69 (t, 2H), 3.57 (m, 4H), 4.12 (t, 2H), 6.99 (d, 2H), 7.11 (d, 1H), 7.28 (s, 1H), 7.56 (d, 2H), 7.67 (d, 1H), 7.92 (s, 1H), 7.99 (s, 1H), 8.50 (d, 1H), 11.92 (bs, 1H), 12.57 (bs, 1H).

MS (ESI⁺) m/z: 444 [MH]⁺

### EXAMPLE 46

### 6-(2-{(E)-2-[4-(2-Hydroxy-2-methyl-propoxy)-phenyl]-vinyl}-pyridin-4-yl)-3,7-dihydro-pyrrolo[2,3-d]pyrimidin-4-one

### a) [4-((E)-2-Iodovinyl)phenoxy]-acetic acid Ethyl Ester

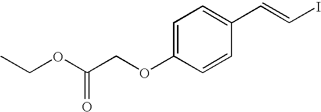

Mixture of 4-((E)-2-iodovinyl)-phenol (1.2 g, 5 mmol), ethyl bromoacetate (1.0 mg, 6 mmol), NaI (75 mg, 0.5 mmol) and K₂CO₃ (1.4 g, 10 mmol) in acetone (50 ml) is stirred at 50° C. for 16 h. After evaporation of the solvent, the mixture is treated with saturated NH₄Cl solution (aq.) and extracted with ethyl acetate. The organic layers are then washed with water and brine, dried over Na₂SO₄ and the solvent is evaporated. The title product is obtained after chromatography purification of the crude product.

¹H-NMR (400 MHZ, CDCl₃): 1.32 (t, 2H), 4.29 (q, 2H), 4.63 (s, 2H), 6.68 (d, 1H), 6.88 (d, 2H), 7.24 (d, 2H), 7.36 (d, 1H).

MS (ESI⁺) m/z: 350 [M+NH₄]⁺

### b) 1-[4-((E)-2-Iodovinyl)phenoxy]-2-methyl-propan-2-ol

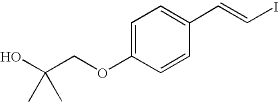

A solution of [4-((E)-2-iodovinyl)-phenoxy]-acetic acid ethyl ester (349 mg, 1.1 mmol) in dry diethylether (2 ml) is added dropwise over 15 min. to a solution of methylmagnesium bromide (1.1 ml, 3M sol. in Et₂O, 3.2 mmol) in Et₂O (1 ml) at −78° C. The mixture is left to react at room temperature for 30 min. After quenching the reaction mixture by addition of saturated NH₄Cl (aq.) the mixture is diluted with Et₂O. 0.2N HCl (aq.) is added until the solution becomes clear and the water phase is extracted with ethyl acetate. Organic layers are then washed with water, brine, dried (Na₂SO₄) and finally the solvent is evaporated to give the title product.

¹H-NMR (400 MHZ, CDCl₃): 1.37 (s, 6H), 2.22 (bs, 1H), 3.82 (s, 2H), 6.67 (d, 1H), 6.89 (d, 2H), 7.29 (d, 2H), 7.38 (d, 1H).

MS (ESI⁺) m/z: 336 [M+NH₄]⁺

### c) 2-Methyl-1-{4-[(E)-2-(4,4,5,5-tetramethyl-[1,3,2]dioxaborolan-2-yl)-vinyl]-phenoxy}-propan-2-ol

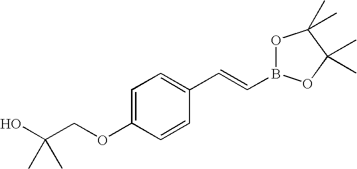

Starting from 1-[4-((E)-2-iodovinyl)phenoxy]-2-methyl-propan-2-ol (300 mg, 0.94 mmol) the desired boronate is obtained as described in example 42b).

¹H-NMR (400 MHZ, CDCl₃): 1.33 (s, 12H), 1.37 (s, 6H), 3.83 (s, 2H), 6.05 (d, 1H), 6.92 (d, 2H), 7.38 (d, 1H), 7.47 (d, 2H).

MS (ESI⁺) m/z: 319 [MH]⁺

### c) 6-(2-{(E)-2-[4-(2-Hydroxy-2-methyl-propoxy)-phenyl]-vinyl}-pyridin-4-yl)-3,7-dihydro-pyrrolo[2,3-d]pyrimidin-4-one

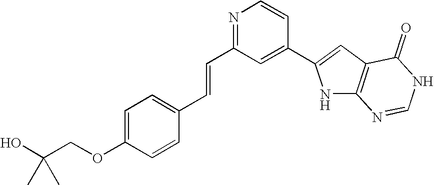

Starting from 6-(2-chloro-pyridin-4-yl)-3,7-dihydro-pyrrolo[2,3-d]pyrimidin-4-one, (76 mg, 0.27 mmol) and 2-methyl-1-{4-[(E)-2-(4,4,5,5-tetramethyl-[1,3,2]dioxaborolan-2-yl)-vinyl]-phenoxy}-propan-2-ol (129 mg, 0.41 mmol), the title compound is prepared as described in example 42c). After chromatography purification the desired product is obtained.

¹H-NMR (400 MHZ, DMSO-d6): 1.21 (s, 6H), 3.75 (s, 2H), 4.63 (s, 1H), 6.97 (d, 2H), 6.99 (d, 1H), 7.12 (d, 1H), 7.32 (s, 1H), 7.56 (d, 2H), 7.65 (d, 1H), 7.92 (s, 1H), 8.03 (s, 1H), 8.50 (d, 1H), 11.93 (bs, 1H), 12.59 (bs, 1H).

MS (ESI⁺) m/z: 403 [MH]⁺

### EXAMPLE 47

### 6-[6′-(4-Methyl-piperazin-1-yl)-[2,3′]bipyridinyl-4-yl]-3,7-dihydro-pyrrolo[2,3-d]pyrimidin-4-one

### a) 1-(5-Bromo-pyridin-2-yl)-4-methyl-piperazine

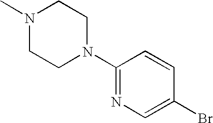

A mixture of 2,5-dibromopyridine (1.0 g, 4.2 mmol) and methyl-piperazine (1.5 ml) is heated to 110° C. for 90 minutes. Excess methyl-piperazine is removed under vacuum and the residue poured onto aqueous NaHCO₃. Extraction with ethylacetate and removal of the solvent yields the desired product.

¹H-NMR (400 MHZ, DMSO-d6): 2.19 (s, 3H), 2.35 (t, 4H), 3.44 (t, 4H), 6.79 (d, 1H), 7.64 (dd, 1H), 8.12 (d, 1H).

MS (ESI⁺) m/z: 256 [MH]⁺

### b) 1-Methyl-4-[5-(4,4,5,5-tetramethyl-[1,3,2]dioxaborolan-2-yl)-pyridin-2-yl]-piperazine

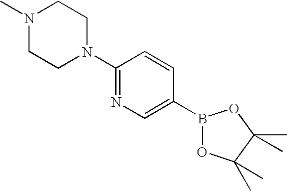

A solution of 1-(5-bromo-pyridin-2-yl)-4-methyl-piperazine (0.52 g, 2.3 mmol) in 15 ml THF is cooled to −78° C. and n-butyllithium (1.7 ml, 1.6 M solution in hexane) is added dropwise. Stirring is continued for 30 minutes after which time 2-isopropoxy-4,4,5,5-tetramethyl-1,3,2-dioxoborolan (511 mg, 2.7 mmol) is added. After 2 hours the mixture is allowed to warm to room temperature and quenched by addition of aq. NaHCO3. Extraction with ethylacetate, drying with Na2SO4 and removal of the solvent gives the desired boronate which was used as crude material in subsequent reactions.

MS (ESI⁺) m/z: 304 [MH]⁺

### c) 6-[6′-(4-Methyl-piperazin-1-yl)-[2,3′]bipyridinyl-4-yl]-3,7-dihydro-pyrrolo[2,3-d]pyrimidin-4-one

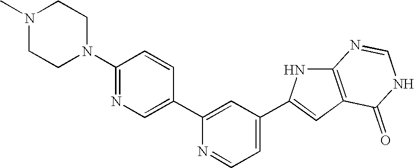

Coupling of 6-(2-Chloro-pyridin-4-yl)-3,7-dihydro-pyrrolo[2,3-d]pyrimidin-4-one (150 mg, 0.61 mmol) with the above described boronate (249 mg, 0.8 mmol) as described in Example 1d) gives the title compound in as white solid.

¹H-NMR (400 MHZ, DMSO-d6): 2.22 (s, 3H), 2.41 (t, 4H), 3.58 (t, 4H), 6.93 (d, 1H), 7.29 (s, 1H), 7.63 (d, 1H), 7.83 (s, 1H), 8.26 (s, 1H), 8.28 (d, 1H), 8.49 (d, 1H), 8.91 (s, 1H), 12.1 (br s, 1H).

MS (ESI⁺) m/z: 388 [MH]⁺

### EXAMPLE 48

### 2-Amino-6-[6′-(4-methyl-piperazin-1-yl)-[2,3′]bipyridinyl-4-yl]-3,7-dihydro-pyrrolo[2,3-d]pyrimidin-4-one

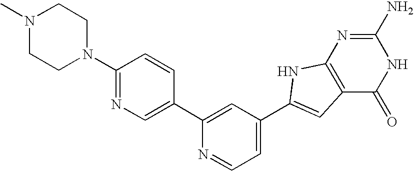

¹H-NMR (400 MHZ, DMSO-d6): 2.22 (s, 3H), 2.40 (t, 4H), 3.57 (t, 4H), 6.27 (s, 2H), 6.92 (d, 1H), 7.15 (s, 1H), 7.51 (d, 1H), 8.14 (s, 1H), 8.26 (d, 1H), 8.90 (s, 1H), 10.4 (s, 1H), 11.7 (s, 1H).

MS (ESI⁺) m/z: 403 [MH]⁺

In analogy the following compounds were prepared:

### EXAMPLE 49

### 6-(6′-Pyrrolidin-1-yl-[2,3′]bipyridinyl-4-yl)-3,7-dihydro-pyrrolo[2,3-d]pyrimidin-4-one

### a) 5-Bromo-2-pyrrolidin-1-yl-pyridine

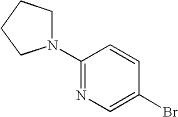

¹H-NMR (400 MHZ, DMSO-d6): 1.89-1.95 (m, 4H), 3.28-3.36 (m, 4H), 6.40 (d, 1H), 7.58 (d, 1H), 8.08 (s, 1H).

MS (ESI⁺) m/z: 227 [MH]⁺

### b) 2-Pyrrolidin-1-yl-5-(4,4,5,5-tetramethyl-[1,3,2]dioxaborolan-2-yl)-pyridine

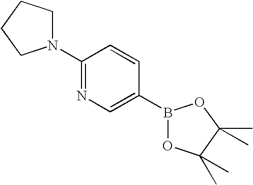

MS (ESI⁺) m/z: 275 [MH]⁺

### c) 6-(6′-Pyrrolidin-1-yl-[2,3′]bipyridinyl-4-yl)-3,7-dihydro-pyrrolo[2,3-d]pyrimidin-4-one

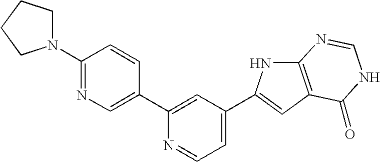

¹H-NMR (400 MHZ, DMSO-d6): 1.97 (br s, 4H), 3.48 (br s, 4H), 6.55 (d, 1H), 7.39 (s, 1H), 7.63 (d, 1H), 7.94 (s, 1H), 8.27 (d, 1H), 8.29 (s, 1H), 8.51 (d, 1H), 8.93 (s, 1H), 12.1-12.7 (m, 2H).

MS (ESI⁺) m/z: 359 [MH]⁺

### EXAMPLE 50

### 6-[6′-(2-Pyrrolidin-1-yl-ethoxy)-[2,3′]bipyridinyl-4-yl]-3,7-dihydro-pyrrolo[2,3-d]pyrimidin-4-one

### a) 5-Bromo-2-(2-pyrrolidin-1-yl-ethoxy)-pyridine

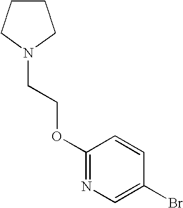

¹H-NMR (400 MHZ, DMSO-d6): 1.63-168 (m, 4H), 2.43-2.50 (m, 4H), 2.74 (t, 2H), 4.30 (t, 2H), 6.80 (d, 1H), 7.85 (dd, 1H), 8.23 (d, 1H).

MS (ESI⁺) m/z: 271 [MH]⁺

### b) 2-(2-Pyrrolidin-1-yl-ethoxy)-5-(4,4,5,5-tetramethyl-[1,3,2]dioxaborolan-2-yl)-pyridine

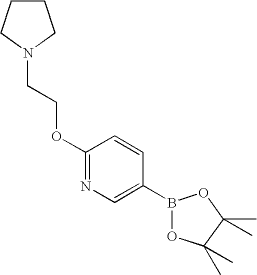

MS (ESI⁺) m/z: 319 [MH]⁺

### c) 6-[6′-(2-Pyrrolidin-1-yl-ethoxy)-[2,3′]bipyridinyl-4-yl]-3,7-dihydro-pyrrolo[2,3-d]pyrimidin-4-one

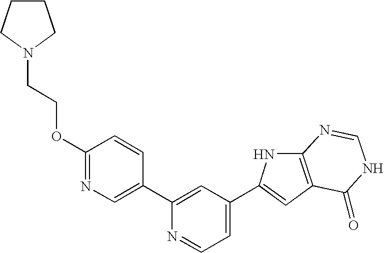

¹H-NMR (400 MHZ, DMSO-d6): 1.69 (s, 4H), 2.55 (s, 4H), 2.84 (t, 2H), 4.43 (t, 2H), 6.93 (d, 1H), 7.43 (s, 1H), 7.75 (d, 1H), 7.93 (s, 1H), 8.38 (s, 1H), 8.44 (dd, 1H), 8.58 (d, 1H), 8.95 (d, 1H), 11.9 (s, 1H), 12.6 (s, 1H).

MS (ESI⁺) m/z: 403 [MH]⁺

### EXAMPLE 51

### 2-Benzyl-6-[2-((E)-styryl)-pyridin-4-yl]-3,7-dihydro-pyrrolo[2,3-d]pyrimidin-4-one

### a) 2-Amino-5-[2-((E)-2-pyridin-3-yl-vinyl)-pyridin-4-yl]-1H-pyrrole-3-carboxylic acid Ethyl Ester

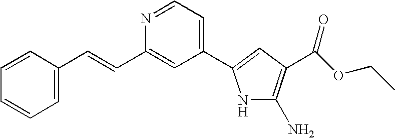

2-Amino-5-(2-chloro-pyridin-4-yl)-1H-pyrrole-3-carboxylic acid ethyl ester (4.04 g, 15.2 mmol), E-phenyl-vinyl boronic acid (2.70 g, 18.2 mmol), 2M Na₂CO₃ (11.4 ml, 22.8 mmol) and Pd(PPh₂)₂Cl₂ (1.10 g, 1.52 mmol) are suspended in 15 ml n-propanol, purged with argon and subjected to microwave heating (140° C., 10 min). The reaction mixture is diluted with ethylacetate, washed with water and brine, dried over Na₂SO₄ and concentrated in vacuo. The crude material is purified by silica gel chromatography (hexanes/ethylacetate) to afford a red solid.

¹H-NMR (400 MHZ, DMSO-d6): 1.27 (t, 3H), 4.16 (q, 2H), 5.89 (s, 2H), 6.89 (d, 1H), 7.21 (d, 1H), 7.28 (dd, 1H), 7.32 (d, 1H), 7.40 (d, 1H), 7.40 (s, 2H), 7.67 (s, 1H), 8.37 (d, 1H) 10.99 (s, 1H).

MS (ESI⁺) m/z: 334 [MH]⁺

### b) 2-Benzyl-6-[2-((E)-styryl)-pyridin-4-yl]-3,7-dihydro-pyrrolo[2,3-d]pyrimidin-4-one

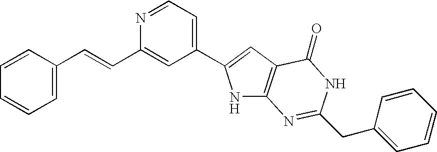

2-Amino-5-[2-((E)-2-pyridin-3-yl-vinyl)-pyridin-4-yl]-1H-pyrrole-3-carboxylic acid ethyl ester 100 mg, 0.30 mmol) and 2-phenyl-acetamidine (211.7 mg, 1.50 mmol) are dissolved in 6 ml N,N-dimethylformamide. To this mixture K₂CO₃ (290.2 mg, 2.10 mmol) is added. The reaction is stirred for 2 days at 80° C. and then the solvent is evaporated under high vacuum. The residue is re-crystallized from hot methanol. The crystals were washed with water and methanol and dried.

¹H-NMR (400 MHZ, DMSO-d6): 5.20 (s, 2H), 7.26 (d, 1H), 7.32-7.35 (m, 6H), 7.35 (s, 1H), 7.41 (dd, 2H), 7.65 (d, 2H), 7.68 (s, 1H), 7.73 (d, 1H), 8.07 (s, 1H), 8.43 (s, 1H), 8.54 (d, 1H), 12.69 (s, 1H).

MS (ESI⁺) m/z: 405 [MH]⁺

### EXAMPLE 52

### 2-Butyl-6-[2-((E)-styryl)-pyridin-4-yl]-3,7-dihydro-pyrrolo[2,3-d]pyrimidin-4-one

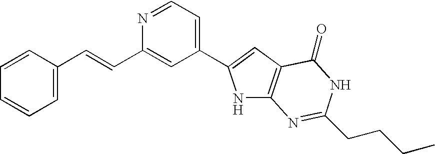

The title compound is prepared as described in example 51) starting from 2-amino-5-[2-((E) 2-pyridin-3-yl-vinyl)-pyridin-4-yl]-1H-pyrrole-3-carboxylic acid ethyl ester (200 mg, 0.60 mmol) and pentaneamidine hydrochloride (409 mg, 3.0 mmol). Pale yellow crystals are obtained.

¹H-NMR (400 MHZ, DMSO-d6): 0.90 (t, 3H), 1.30-1.40 (m, 2H), 1.65-1.75 (m, 2H), 2.60-2.65 (m, 2H), 7.25 (t, 2H), 7.33 (d, 1H), 7.38-7.45 (m, 2H), 7.65 (s, 1H), 7.66 (s, 1H), 7.68 (d, 1H), 7.73 (d, 1H), 8.04 (s, 1H), 8.52 (s, 1H), 11.84 (s, 1H), 12.51 (s, 1H).

MS (ESI⁺) m/z: 371 [MH]⁺

### EXAMPLE 53

### 2-Cyclopropyl-6-[2-((E)-styryl)-pyridin-4-yl]-3,7-dihydro-pyrrolo[2,3d]pyrimidin-4-one

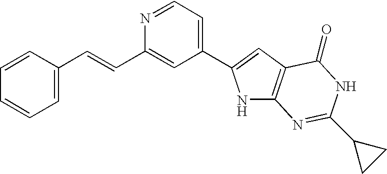

The title compound is prepared as described in example 51) starting from 2-amino-5-[2-((E) 2-pyridin-3-yl-vinyl)-pyridin-4-yl]-1H-pyrrole-3-carboxylic acid ethyl ester (100 mg, 0.30 mmol) and cyclopropylcarbamidine hydrochloride (190 mg, 1.5 mmol). Pale yellow crystals are obtained.

¹H-NMR (400 MHZ, DMSO-d6): 1.04 (s, 4H), 1.96-2.06 (m, 1H), 7.22 (s, 1H), 7.23 (d, 1H), 7.31 (t, 1H), 7.40 (t, 2H), 7.61 (d, 1H), 7.64 (d, 2H), 7.68 (d, 1H), 8.01 (s, 1H), 8.47 (d, 1H), 12.10 (s, 1H), 12.36 (s, 1H).

MS (ESI⁺) m/z: 355 [MH]⁺

### EXAMPLE 54

### 2-Methylsulfanyl-6-[2-((E)-styryl)-pyridinyl]-3,7-dihydro-pyrrolo[2,3-d]pyrimidin-4-one

### a) 5-[2-((E)-Styryl)-pyridin-4-yl]-2-ethoxycarbonyl-thioureido-1H-pyrrole-3-carboxylic acid Ethyl Ester

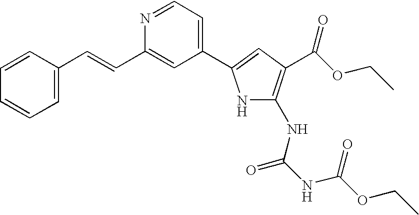

2-Amino-5-[2-((E)-2-pyridin-3-yl-vinyl)-pyridin-4-yl]-1H-pyrrole-3-carboxylic acid ethyl ester (1.20 g, 3.60 mmol) is dissolved in 100 ml of toluene and ethoxycarbonylisothiocyanate (0.60 g, 4.53 mmol) is slowly added at r.t. The resulting solution is heated to 95° C. for 17 h. When cooling down to r.t. the product precipitates. The orange crystals are collected by filtration, washed with hexanes and dried.

¹H-NMR (400 MHZ, DMSO-d6): 1.29 (t, 3H), 1.31 (t, 3H), 4.25 (q, 2H), 4.27 (q, 2H), 7.27 (d, 1H), 7.32 (s, 1H), 7.33 (d, 1H), 7.41 (dd, 2H), 7.44 (d, 1H), 7.66 (d, 2H), 7.73 (d, 1H), 7.81 (s, 1H), 8.51 (s, 1H), 11.77 (s, 1H), 12.88 (s, 1H), 12.91 (s, 1H).

MS (ESI⁺) m/z: 465 [MH]⁺

### b) 6-[2-((E)-Styryl)-pyridin-4-yl]-2-thioxo-1,2,3,7-tetrahydro-pyrrolo[2,3-d]pyrimidin-4-one

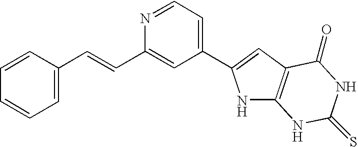

A solution of 5-[2-((E)-styryl)-pyridin-4-yl]-2-ethoxycarbonyl-thioureido-1H-pyrrole-3-carboxylic acid ethyl ester (1.40 g, 3.01 mmol) in 35 ml of aqueous KOH (6%) is heated to reflux for 20 h. After cooling to 0° C. the solution is acidified by HCl conc. The precipitate is filtered, washed with water and hexanes and dried. The product is used in the next step without further purification.

### c) 2-Methylsulfanyl-6-[2-((E)-styryl)-pyridin-4-yl]-3,7-dihydro-pyrrolo[2,3-d]pyrimidin-4-one

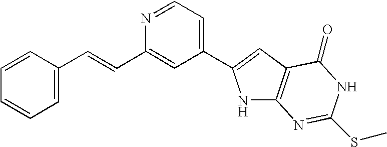

6-[2-((E)-Styryl)-pyridin-4-yl]-2-thioxo-1,2,3,7-tetrahydro-pyrrolo[2,3-d]pyrimidin-4-one (2.20 g, 5.75 mmol) and K₂CO₃ (2.0 g, 14.47 mmol) are suspended in 120 ml of acetone and iodomethane (0.50 ml, 8.10 mmol) is slowly added at r.t. After 3 h the mixture is filtered and evaporated. Purification of the crude product by flash chromatography (silica gel, ethylacetate/methanol) gives colorless crystalline material.

¹H-NMR (400 MHZ, DMSO-d6): 2.58 (s, 3H), 7.24 (d, 1H), 7.24 (s, 1H), 7.32 (dd, 1H), 7.42 (dd, 2H), 7.64 (s, 1H), 7.65 (d, 2H), 7.71 (d, 1H), 8.05 (s, 1H), 8.51 (s, 1H), 12.22 (s, 1H), 12.48 (s, 1H).

MS (ESI⁺) m/z: 361 [MH]⁺

### EXAMPLE 55

### 2-(2,3-Dihydroxy-propylamino)-6-[2-((E)-styryl)-pyridin-4-yl]-3,7-dihydro-pyrrolo[2,3-d]pyrimidin-4-one

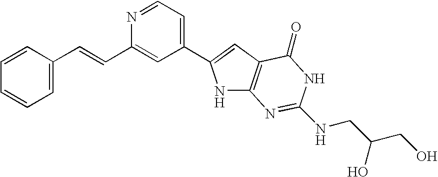

2-Methylsulfanyl-6-[2-((E)-styryl)-pyridin-4-yl]-3,7-dihydro-pyrrolo[2,3-d]pyrimidin-4-one (100 mg, 0.277 mmol) is dissolved in 2 ml 3-amino-propane-1,2-diol and heated for 4 h at 170° C. The reaction mixture is diluted with ethylacetate, washed with water and brine and concentrated in vacuo. The brown residue is purified on silica gel (ethylacetate/methanol) and by reverse phase HPLC (Waters X-Terra, acetonitrile/water).

¹H-NMR (400 MHZ, DMSO-d6): 3.16-3.20 (m, 1H), 3.34-3.36 (m, 1H), 3.40-3.42 (m, 1H), 3.52-3.54 (m, 1H), 3.63-3.65 (m, 1H), 4.68 (brs, 1H), 5.00 (brs, 1H), 6.41 (t, 1H), 7.10 (s, 1H), 7.22 (d, 1H), 7.32 (dd, 1H), 7.41 (dd, 2H), 7.54 (s, 1H), 7.64 (d, 2H), 7.70 (d, 1H), 7.94 (s, 1H), 8.43 (d, 1H), 10.37 (brs, 1H), 11.90 (s, 1H).

MS (ESI⁺) m/z: 304 [MH]⁺

### EXAMPLE 56

### 2-(2-Hydroxy-1-hydroxymethyl-ethylamino)-6-[2-((E)-styryl)-pyridin-4-yl]-3,7-dihydro-pyrrolo[2,3-d]pyrimidin-4-one

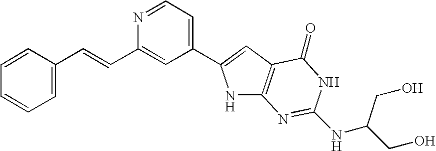

The title compound is prepared as described in example 55) starting from 2-methylsulfanyl-6-[2-((E)-styryl)-pyridin-4-yl]-3,7-dihydro-pyrrolo[2,3-d]pyrimidin-4-one (100 mg, 0.277 mmol) and serinol (2 ml). Orange crystals are obtained.

¹H-NMR (400 MHZ, DMSO-d6): 3.50-3.62 (m, 4H), 3.83-3.89 (m, 1H), 4.85 (brs, 2H), 6.42 (brs, 1H), 7.10 (s, 1H), 7.22 (d, 1H), 7.32 (dd, 1H), 7.41 (dd, 2H), 7.54 (dm, 1H), 7.64 (d, 2H), 7.70 (d, 1H), 7.96 (s, 1H), 8.41 (d, 1H), 10.35 (brs, 1H), 11.90 (s, 1H).

MS (ESI⁺) m/z: 404 [MH]⁺

### EXAMPLE 57

### 6-[2-(4-Benzyloxy-phenyl)-pyridinyl]-3,7-dihydro-pyrrolo[2,3-d]pyrimidin-one

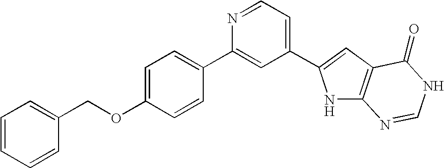

4-(Phenylmethoxy)benzeneboronic acid (277 mg) and 6-(2-chloro-pyridin-4-yl)-3,7-dihydro-pyrrolo[2,3-d]pyrimidin-4-one (100 mg), are dissolved in 4 ml dioxane. The solution is degassed by introduction of a stream of argon, Pd(PPh₂)₂Cl₂ (47 mg), and 258 mg of potassium triphosphate are added. The mixture is heated for 10 minutes in a microwave oven at 180° C. The reaction mixture is filtered over celite and dried. The residual yellow oil (200 mg) is purified by flash chromatography (ethyl acetate/cyclohexane).

¹H-NMR (400 MHZ, DMSO-d6): 5.18 (s, 2H), 7.14 (d, 2H) 7.32 (m, 1H), 7.39 (m, 3H), 7.46 (d, 2H), 7.69 (bs, 1H), 7.92 (s, 1H), 8.14 (d, 2H), 8.33 (s, 1H), 8.54 (d, 1H), 11.91 (bs, 1H), 12.59 (bs, 1H).

MS (ESI⁺) m/z: 395 [MH]⁺

### EXAMPLE 58

### N-Cyclopentyl-4-[4-(4-oxo-4,7-dihydro-3H-pyrrolo[2,3-d]pyrimidin-6-yl)-pyridin-2-yl]-benzamide

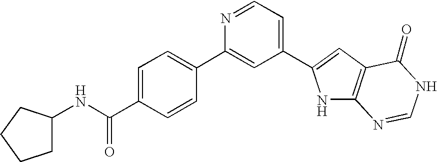

4-(4,4,5,5-tetramethyl-1,3,2-dioxaborolan-2-yl)benzoic acid (514 mg) and 0.23 ml of cyclopentylamin are dissolved in dichloromethane. After addition of 1-(3-Dimethylaminopropyl)-3-ethylcarbodiimide hydrochloride (463 mg) and 1-Hydroxybenzotriazole (326 mg), The reaction is stirred at room temperature. The reaction mixture is diluted with ethyl acetate washed with 1N hydrochloric acid/brine and dried over sodium sulfate. After evaporation of the solvent the residual yellow solid 800 mg is purified by flash chromatography (ethyl acetate/hexane:1/9).

160 mg of N-Cyclopentyl-4-(4,4,5,5-tetramethyl-[1,3,2]dioxaborolan-2-yl)benzamide and 50 mg of 6-(2-Chloro-pyridin-4-yl)-3,7-dihydro-pyrrolo[2,3-d]pyrimidin-4-one are dissolved in 2 ml dimethylformamide. The solution is degassed by introduction of a stream of argon, Pd(PPh₂)₂Cl₂ (14 mg), and 0.5 ml of sodium carbonate 2N are added. The mixture is heated for 15 min in a microwave oven at 160° C. The reaction mixture is diluted with ethyl acetate washed with water/brine. After being dried over sodium sulfate, the residue is purified by HPLC (acetonitrile/water, RP-18).

MS (ESI⁻) m/z: 398 [M−H]⁻

The following examples are synthesised in analogy:

### EXAMPLE 59

### N-(4-Hydroxy-cyclohexyl)-4-[4-(4-oxo-4,7-dihydro-3H-pyrrolo[2,3-d]pyrimidin-6-yl)-pyridin-2-yl]-benzamide

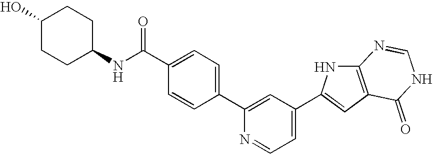

MS (ESI⁺) m/z: 430 [MH]^{+/MS (ESI}⁻) m/z: 428 [M−H]⁻

### EXAMPLE 60

### N-[2-(2-Methoxy-ethoxy)-ethyl]-4-[4-(4-oxo-4,7-dihydro-3H-pyrrolo[2,3-d]pyrimidin-6-yl)-pyridin-2-yl]-benzamide

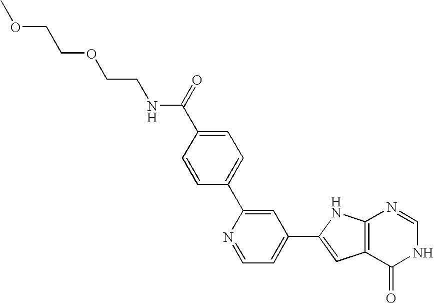

¹H-NMR (400 MHZ, DMSO-d6): 3.23 (s, 3H), 3.42-3.50 (m, 4H), 3.52-3.60 (m, 4H), 7.44 (s, 1H), 7.80 (d, 1H), 7.93 (s, 1H), 7.97 (d, 2H), 8.28 (d, 2H), 8.48 (s, 1H), 8.60 (t, 1H), 8.63 (d, 1 HO, 11.8-12.7 (broad signal, 2H)

MS (ESI⁺) m/z: 434 [MH]⁺

### EXAMPLE 61

### 2-(3-Methyl-butylamino)-6-[2-((E)-styryl)-pyridin-4-yl]-3,7-dihydro-pyrrolo[2,3-d]pyrimidin-4-one

### a) 6-(2-Chloro-pyridin-4-yl)-2-(3-methyl-butylamino) 3,7-dihydro-pyrrolo[2,3-d]pyrimidin-4-one

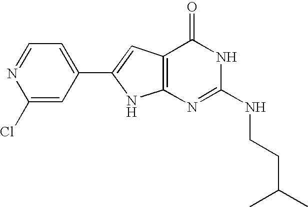

2-Amino-6-(2-chloro-pyridin-4-yl)-3,7-dihydro-pyrrolo[2,3-d]pyrimidin-4-one (150 mg, 0.6 mmol) and 3-methyl-butyraldehyde (69 mg, 0.8 mmol) are dissolved in DMF/acetic acid 97:3 and are stirred at room temperature for 20 minutes, then sodium cyanoborohydride (72 mg, 1.1 mmol) is added. The reaction mixture is stirred for additional 72 hours, then quenched by the addition of 1N HCl. The mixture is brought to pH 9 using NaHCO3 and extracted with ethyl acetate. The crude product obtained after drying and evaporation of the solvent is purified by HPLC to yield the desired product.

¹H-NMR (400 MHZ, DMSO-d6): 0.91 (d, 6H), 1.43 (q, 2H), 1.64 (m, 1H), 3.25-3.33 (m, 2H), 6.26 (t, 1H), 7.17 (s, 1H), 7.67 (d, 1H), 7.79 (s, 1H), 8.22 (d, 1H), 10.2 (s, 1H), 11.9 (s, 1H).

MS (ESI⁺) m/z: 332 [MH]⁺

### b) 2-(3-Methyl-butylamino)-6-[2-((E)-styryl)-pyridin-4-yl]-3,7-dihydro-pyrrolo[2,3-d]pyrimidin-4-one

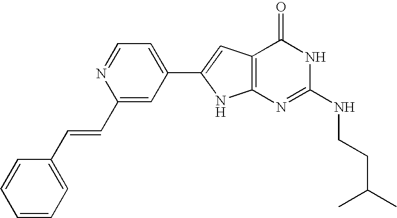

Suzuki coupling as described in example 1d) provides the title compound.

¹H-NMR (400 MHZ, DMSO-d6): 0.94 (d, 6H), 1.40-1.52 (m, 2H), 1.60-1.72 (m, 1H), 2.42-2.57 (m, 2H), 6.25 (s, 1H), 7.09 (s, 1H), 7.21 (d, 1H), 7.32 (t, 1H), 7.41 (t, 2H), 7.55 (d, 1H), 7.65 (d, 2H), 7.68 (d, 1H), 7.96 (s, 1H), 8.44 (d, 1H), 10.23 (s, 1H), 11.88 (s, 1H).

MS (ESI⁺) m/z: 400 [MH]⁺

### EXAMPLE 62

### 2-(2-Hydroxy-ethylamino)-6-[2-((E)-styryl)-pyridin-4-yl]-3,7-dihydro-pyrrolo[2,3-d]pyrimidin-4-one

### a) 2-[2-tert-Butyl-dimethyl-silanyloxy)-ethylamino]-6-[2-((E)-styryl)-pyridin-4-yl]-3,7-dihydro-pyrrolo[2,3-d]pyrimidin-4-one

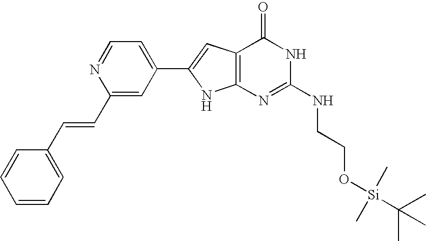

Reductive amination using 2-amino-6-[2-((E)-styryl)-pyridin-4-yl]-3,7-dihydro-pyrrolo[2,3-d]pyrimidin-4-one (example 4) (150 mg, 0.5 mmol) and tert, butyldimethylsilyloxy acetaldehyde (111 mg, 0.6 mmol) as described in example 61), provides the title compound.

¹H-NMR (400 MHZ, DMSO-d6): 0.07 (s, 6H), 0.89 (s, 9H), 3.42 (q, 2H), 3.75 (t, 2H), 6.24 (t, 1H), 7.10 (s, 1H), 7.21 (d, 1H), 7.32 (t, 1H), 7.42 (t, 2H), 7.55 (d, 1H), 7.64 (d, 2H), 7.69 (d, 1H), 7.95 (s, 1H), 8.43 (d, 1H), 10.5 (s, 1H), 11.9 (s, 1H).

MS (ESI⁺) m/z: 488 [MH]⁺

### b) 2-(2-Hydroxy-ethylamino)-6-[2-((E)-styryl)-pyridin-4-yl]-3,7-dihydro-pyrrolo[2,3-d]pyrimidin-4-one

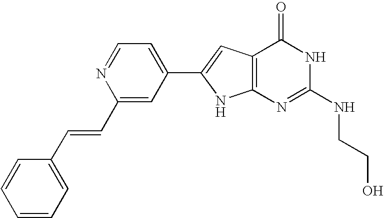

2-[2-(tert-Butyl-dimethyl-silanyloxy)ethylamino]-6-[2-((E)-styryl)-pyridin-4-yl]-3,7-dihydro-pyrrolo[2,3-d]pyrimidin-4-one (8.0 mg) is dissolved in ethanol/dioxan (1 ml). Two drops of conc. HCl are added and the mixture is stirred at room temperature for 2 hours. Removal of the solvent and tituration of the residue with ether gives the desired alcohol.

MS (ESI⁺) m/z: 374 [MH]⁺

### EXAMPLE 63

### 3-(2-Methoxy-ethyl)-6-[2-((E)-styryl)-pyridin-4-yl]-3,7-dihydro-pyrrolo[2,3-d]pyrimidin-4-one

### a) 6-(2-Chloro-pyridin-4-yl)-3-(2-methoxy-ethyl)-3,7-dihydro-pyrrolo[2,3-d]pyrimidin-4-one

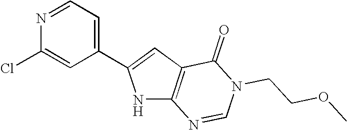

To a mixture of sodium hydride (60% in mineral oil, 101 mg, 2.5 mmol) in ml DMF is added 6-(2-chloro-pyridin-4-yl)-3,7-dihydro-pyrrolo[2,3-d]pyrimidin-4-one (250 mg, 1.0 mmol) in DMF (2 ml). The reaction mixture is stirred at room temperature for 20 minutes, then 2-chloroethyl methylether (115 mg, 1.2 mmol) is added. After additional stirring for 20 hours the reaction is stopped by addition of aq. NaHCO3 solution. The mixture is extracted with ethylacetate, the organic phase dried and evaporated. The crude product obtained was sufficiently pure to be used in further reactions.

¹H-NMR (400 MHZ, DMSO-d6): 3.27 (s, 3H), 3.60 (t, 2H), 4.17 (t, 2H), 7.42 (s, 1H), 7.85 (d, 1H), 7.96 (s, 1H), 8.16 (s, 1H), 8.48 (d, 1H), 12.7 (s, 1H).

MS (ESI⁺) m/z: 305 [MH]⁺

### b) 3-(2-Methoxy-ethyl)-6-[2-((E)-styryl)-pyridin-4-yl]-3,7-dihydro-pyrrolo[2,3-d]pyrimidin-4-one

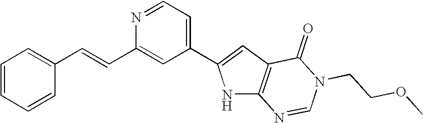

Suzuki coupling as described in example 1d) yields the desired compound.

¹H-NMR (400 MHZ, DMSO-d6): 3.27 (s, 3H), 3.60 (t, 2H), 4.17 (t, 2H), 7.28 (d, 1H), 7.34 (t, 1H), 7.36 (s, 1H), 7.42 (t, 2H), 7.67 (d, 2H), 7.69 (d, 1H), 7.73 (d, 1H), 8.08 (s, 1H), 8.15 (s, 1H), 8.54 (d, 1H), 12.6 (s, 1H).

MS (ESI⁺) m/z: 373 [MH]⁺

Agents of the Invention possess MAPKAPK2 (MAP Kinase Activated Protein Kinase) inhibiting activity. Thus the Agents of the Invention act to inhibit production of inflammatory cytokines, such as TNF-α, and also to potentially block the effects of these cytokines on their target cells. These and other pharmacological activities of the Agents of the Invention as may be demonstrated in standard test methods for example as described below:

### MAPKAPK2 Kinase Assay

MAPKAPK2 is pre-activated in kinase buffer (25 mM TRIS-HCL, pH 7.5, 25 mM beta-glycerophosphate, 0.1 mM sodium orthovanadate, 25 mM MgCl₂, 20 μM DTT) containing 5 μM ATP, 150 μg/ml human MK2 (HPLC purified in house), 30 μg/ml active human p38α (HPLC purified in house) for 30 min at 22° C. For the measurement of compound inhibition on activated MAPKAPK2, each reaction contained test compound (10 μl; 0.5% DMSO final) or vehicle control, 250 nM Hsp27 peptide biotinyl-AYSRALSRQLSSGVSEIR-COOH as substrate (10 μl) and pre-activated MAPKAPK2 kinase mix (10 μl) containing ATP (5 μM final). To define non-specific, reactions are performed in the absence of substrate. Following incubation at 22° C. for 45 min, kinase reactions are terminated with 125 μM EDTA (10 μl).

Samples (10 μl) are transferred to black low volume 384-well plates (Greiner) prior to the detection of phosphorylated substrate by time-resolved fluorescence resonance energy transfer (TR-FRET). Phosphorylated Hsp27 is measured using an antibody mix (10 μl) containing a rabbit anti-phospho-Hsp27 (Ser⁸²) antibody (2.5 nM, Upstate) in conjunction with an anti-rabbit europium-labeled secondary antibody LANCE Eu-W1024 (2.5 nM; Perkin Elmer) as fluorescence donor along with streptavidin SureLight-APC (6.25 nM; Perkin Elmer) as a fluorescence acceptor. Following incubation at 22° C. for 90 min, plates are measured at 615 and 665 nm using a PHERAstar (BMG Labtech). The 615/665 nm ratio is determined following subtraction of background. Values are expressed as % inhibition using control values. Individual IC₅₀ values of compounds are determined by nonlinear regression after fitting of curves to the experimental data using Excel XL fit 4.0 (Microsoft).

Agents of the invention typically inhibit MK2 activity with IC₅₀ of 0.01 to 10 μM when tested in this assay.

Assay for Inhibition of TNF-α Release from hPBMCs

Human peripheral blood mononuclear cells (hPBMCs) are prepared from the peripheral blood of healthy volunteers using ficoll-hypaque density separation according to the method of Hansell et al., J. Imm. Methods (1991) 145: 105. and used at a concentration of 10⁵ cells/well in RPMI 1640 plus 10% FCS. Cells are incubated with serial dilutions of the test compounds for 30 minutes at 37° C. prior to the addition of IFNg (100 U/ml) and LPS (5 mg/ml) and subsequently further incubated for three hours. Incubation is terminated by centrifugation at 1400 RPM for 10 min. TNF-α in the supernatant is measured using a commercial ELISA (Innotest hTNFa, available from Innogenetics N.V., Zwijnaarde, Belgium). Agents of the Invention are tested at concentrations of from 0 to 10 mM. Exemplified Agents of the Invention typically suppress TNF release in this assay with an IC₅₀ of from about 10 μM to about 10 nM or less when tested in this assay.

### Assay for Inhibition of TNF-α Production in LPS Stimulated Mice

Injection of lipopolysaccharide (LPS) induces a rapid release of soluble tumour necrosis factor (TNF-α) into the periphery. This model is be used to analyse prospective blockers of TNF release in vivo.

LPS (20 mg/kg) is injected i.v. into OF1 mice (female, 8 week old). One (1) hour later blood is withdrawn from the animals and TNF levels are analysed in the plasma by an ELISA method using an antibody to TNF-α. Using 20 mg/kg of LPS levels of up to 15 ng of TNF-α/ml plasma are usually induced. Compounds to be evaluated are given either orally, i.p. or s.c. 1 to 4 hours prior to the LPS injection. Inhibition of LPS-induced TNF-release is taken as the readout.

Agents of the Invention typically inhibit TNF production to the extent of up to about 50% or more in the above assay when administered at 30 mg/kg p.o., or parenterally.

### JAK-3 Kinase Assay

JAK-3 enzymatic activity is determined using a time-resolved fluorescence energy transfer technology. The phosphorylation of a synthetic biotinylated peptide substrate (GGEEEYFELVKKKK) by JAK-3 in the presence of ATP is quantified using Europium labeled anti phosphotyrosine antibody and Streptavidin-Allophycocyanin. The JAK-3 enzyme used in this assay contains the kinase domain (JH-1 domain) of the full length protein and is used as GST fusion protein.

Inhibitors are dissolved in DMSO. Dilutions are prepared in 90% DMSO followed by additional dilutions steps as required to perform a 8-point concentration-response. The reaction mix consists of 5 μL of diluted compound, 10 μL of assay buffer and 5 μL of enzyme dilution. After incubation for 60 minutes at room temperature the reaction is stopped by the addition of EDTA. For detection of the product anti-phosphotyrosine antibody and Streptavidin-APC are added and after 60 minutes the samples are measured in an EnVision 2102 Multilabel Reader with excitation wavelength of 320 nm and emission at 665 nm.

### In Vivo Transplantation

Heterotopic heart allotransplantation in the strain combination DA (donor) to Lewis (recipient) is performed according to standard transplantation procedure. Graft function is monitored by daily palpation of the beating donor heart through the abdominal wall. Rejection is considered to be complete when heart beat stops. Prolongation of graft survival is obtained in animals treated with a compound of formula I administered orally at a daily dose of 1 to 100 mg/kg bid.

Agents of the invention are useful for the prevention and/or treatment of diseases, conditions and disorders that are mediated by TNF alpha and/or by MK2, including autoimmune diseases, inflammation and arthritis, e.g. rheumatoid arthritis. The agents of the invention may also be used for example for the treatment of pain, headaches, or as an antipyretic for the treatment of fever.

In preferred uses, the agents of the invention may be used for the treatment of any of one or more of the following disorders: connective tissue and joint disorders, neoplasia disorders, cardiovascular disorders, ophthalmic disorders, septic shock, respiratory disorders, gastrointestinal disorders, angiogenesis-related disorders, autoimmune and immunological disorders, allergic disorders, infectious diseases and disorders, endocrine disorders, metabolic disorders, neurological and neurodegenerative disorders, pain, hepatic and biliary disorders, musculoskeletal disorders, genitourinary disorders, gynaecological and obstetric disorders, injury and trauma disorders, muscle disorders, surgical disorders, dental and oral disorders, sexual dysfunction orders, dermatological disorders, hematological disorders, and poisoning disorders.

In other preferred embodiments, agents of the invention may be used for the prevention and treatment of autoimmune and inflammatory disorders such as arthritis, e.g. rheumatoid arthritis, psoriatic arthritis, juvenile chronic arthritis, reactive arthritis, arthritis deformans, gouty arthritis, osteoarthritis, atheriosclerosis, restenosis, fibrosis (especially pulmonary, but also other types of fibrosis, such as renal fibrosis), vascular occlusion due to vacular injury such as angioplasty, lyme disease, autoimmune haematological disorders (e.g. hemolytic anaemia, aplastic anaemia, pure red cell anaemia and idiopathic thrombocytopenia), enterogenic spondyloarthropathies, ankylosing spondylitis, inflammatory bowel disease, ulcerative colitis, Crohn's disease, necrotizing enterocolitis, multiple sclerosis, lumbar spondylarthrosis, carpal tunnel syndrome, canine hip dysplasia, systemic lupus erythematosus, lupus nephritis, polychondritis, scleroderma, Sjogre Wegener granulamatosis, glomerulonephritis, nephrotic syndrome, steroid dependent and steroid-resistant nephrosis, palmoplantar pustulosis, allergic encephalomyelitis, Steven-Johnson syndrome, dermatomyositis, polymyositis, Guillain-Barre syndrome, Meniere's disease, radiculopathy, gout, tendonitis and bursitis, organ or transplant rejection (e.g for the treatment of recipients of heart, lung, combined heart lung, liver, kidney, pancreatic, skin or corneal transplants), graft-versus-host disease, sepsis, septic shock, Behcet's disease, uveitis (anterior and posterior), conjunctivitis, keratitis, conical cornea, Sjoegren's syndrome, dystorphia epithelialis corneae, keratoleukoma, vernal conjunctivitis, Muckle-Wells syndrome, psoriasis, cutaneous lupus erythematosus, Hashimoto's thyroiditis, dermatitis, atopic dermatitis, acne vulgaris, alopecia areata, eosinophilic fascitis, eczema, lichen planus, pemphigus, Mooren's ulcer, scleritis, bullous pemphigoid, epidermolysis bullosa, urticaria, angioedemas, vasculitides, cutaneous eosinophilias, xerosis, type I diabetes, Graves disease, Sjogrens syndrome, blistering disorders (e.g. pemphigus vulgaris), chronic autoimmune liver diseases, e.g. autoimmune hepatitis, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, primary biliary cirrhosis and sclerosing cholangitis, rheumatic fever, sarcoidosis, fibroid lung.

In other preferred embodiments be agents of the invention may be used for the prevention and treatment of organ or tissue allo- or xenografts rejection, e.g. acute or chronic rejection of organ or tissue allo- or xenografts, graft-versus-host disease, host-versus-graft disease, e.g for the treatment of recipients of heart, lung, combined heart lung, liver, kidney, pancreatic, skin or corneal transplants.

In other preferred embodiments be agents of the invention may be used for the prevention and treatment of neoplasia disorders such as acral lentiginous melanoma, actinic keratoses, adenocarcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumours, batholin gland carcinoma, basal cell carcinoma, bile duct cancer, bladder cancer, brainstem glioma, brain tumours, breast cancer, bronchial gland carcinomas, capillary carcinoma, carcinoids, carcinoma, carcinomasarcoma, cavernous, central nervous system lymphoma, cerebral astrocytoma, cholangiocarcinoma, chondrosarcoma, choroid plexus papilloma/carcinoma, clear cell carcinoma, skin cancer, brain cancer, colon cancer, colorectal cancer, cutaneous T-cell lymphoma, cystadenoma, endodermal sinus tumour, endometrial hyperplasia, endometrial stromal sarcoma, endometrioid adenocarcinoma, ependymal, epitheloid, esophageal cancer, Ewing's sarcoma, extragonal germ cell tumour, fibrolamellar, focal nodular hyperplasia, gallbladder cancer, gastrinoma, germ cell tumours, gestation trophoblastic tumour, glioblastoma, hemangioblastomas, hemangiomas, hepatic adenomas, hepatic adenomatosis, hepatocellular carcinoma, Hodgkin's lymphoma, hypopharyngeal cancer, hypothalamic and visual pathway glioma, insulinoma, intraepithelial neoplasia, interepithelial cell carcinoma, Kaposi's sarcoma, kidney cancer, laryngeal cancer, leiomyosarcoma, lentigo maligna melanomas, leukaemia-related disorders, lip and oral cavity cancer liver cancer, lung cancer, lymphoma, malignant mesothelial tumours, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, proliferative diseases, myeloproliferative disorders, nasal cavity and paranasal sinus cancer, nasopharyngeal cancer, neuroblastoma, and neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell cancer, pancreatic cancer, papillary serous adenocarcinoma, pineal cell, pituitary tumours, plasmacytoma, pseudosarcoma, pulmonary blastoma, parathyroid cancer, penile cancer, pheochromcytoma, dermal tumours, pituitary tumour, plasma cell neoplasm, pleuropulmonay blastoma, prostate cancer, rectal cancer, renal cell carcinoma, retinoblastoma, rhabdomysarcoma, sarcoma, serous carcinoma, small cell carcinoma, small intestine cancer, soft tissue carcinomas, somatostatin secreting tumour, squamous carcinoma, squamous cell carcinoma, submesothelial, superficial spreading carcinoma, supratentorial primitive neurectodermal tumours, thyroid cancer, undifferentiated carcinoma, urethral cancer, uterine sarcoma, uveal melanoma, verrucous carcinoma, vaginal cancer, vipoma, vulvar cancer, Waldonstrom's macroglobulinemia, well differentiated carcinoma, and Wilm's tumour.

Agents of the invention may further be used to treat or prevent cardiovascular disorders, for example myocardial ischaemia, hypertension, hypotension, heart arrhythmias, pulmonary hypertension, hypokalaemia, cardiac ischaemia, myocardial infarction, cardiac remodelling, cardiac fibrosis, myocardial necrosis, aneurysm, arterial fibrosis, embolism, vascular plaque inflammation, vascular plaque rupture, gut ischemia, bacterial induced inflammation and viral induced inflammation, oedema, swelling, fluid accumulation, cirrhosis of the liver, Bartter's syndrome, myocarditis, arteriosclerosis, at atherosclerosis, calcification (such as vascular calcification and valvar calcification), coronary artery disease, acute coronary syndrome, heart failure, congestive heart failure, shock, arrhythmia, left ventricular hypertrophy, angina, diabetic nephropathy, kidney failure, eye damage, vascular diseases, migraine headaches, aplastic anaemia, cardiac damage, diabetic cardiac myopathy, renal insufficiency, renal injury, renal arteriography, peripheral vascular disease, left ventricular hypertrophy, cognitive dysfunction, stroke and headache.

In other preferred embodiments, agents of the invention may be used for the prevention and treatment of bone and muscle disorders such as sarcopenia, muscular dystrophy, cachexia or wasting syndrome associated with morbid TNF release (e.g. consequent to infection, cancer or organ dysfunction, especially AIDS-related cachexia), and osteoporosis.

In further preferred embodiments agents of the invention may be used for the prevention and treatment of respiratory disorders such as asthma and bronchitis, chronic obstructive pulmonary disease (COPD), cystic fibrosis, pulmonary oedema, pulmonary embolism, pneumonia, pulmonary sarcoisis, silicosis, pulmonary fibrosis, respiratory failure, acute respiratory distress syndrome, primary pulmonary hypertension and emphysema.

In further preferred embodiments, agents of the invention may be used for the prevention and treatment of the angiogenesis-related disorders selected from: angiofibroma, neovascular glaucoma, arteriovenous malformations, arthritis, Osler-Weber syndrome, atherosclerotic plaques, psoriasis, corneal graft neovascularisation, pyogenic granuloma, delayed wound healing, retrolental fibroplasias, diabetic retinopathy, stroke, cancer, AIDS complications, ulcers and infertility.

In further preferred embodiments, agents of the invention may be used for the prevention or treatment of infectious diseases and disorders such as viral infections, bacterial infections, prion infections, spiroketes infections, mycobacterial infections, rickettsial infections, chlamydial infections, parasitic infections and fungal infections.

In yet other preferred embodiments, agents of the invention may be used to the prevention and treatment of neurological and neurodegenerative disorders such as headaches, migraine, pain, dental pain, neuropathic and inflammatory pain, Alzheimer's disease, amyotrophic lateral sclerosis, Parkinson's disease, dementia, memory loss, senility, amyotrophy, ALS, amnesia, seizures, multiple sclerosis, muscular dystrophy use, epilepsy, schizophrenia, depression, anxiety, attention deficit disorder, hyperactivity, spongiform encephalopathy, Creutzfeld-Jacob disease, Huntington's Chorea, ischaemia.

For all the above uses, an indicated daily dosage is in the range from about 0.03 to about 300 mg preferably 0.03 to 30, more preferably 0.1 to 10 mg of a compound of the invention. Agents of the Invention may be administered twice a day or up to twice a week.

The Agents of the Invention may be administered in free form or in pharmaceutically acceptable salt form. Such salts may be prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in form of injectable solutions, microemulsions or suspensions, enterally, e.g. orally, for example in the form of tablets, capsules or drinking solutions; sub-lingual, topically or transdermally, e.g. in form of a dermal cream or gel or for the purpose of administration to the eye in the form of an ocular cream, gel or eye-drop preparation, or it may be administered by inhalation.

The compounds of the invention may also be administered simultaneously, separately or sequentially in combination with one or more other suitable active agents selected from the following classes of agents: Anti IL-1 agents, e.g: Anakinra; anti cytokine and anti-cytokine receptor agents, e.g. anti IL-6R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; gold salts, penicillamine, hydroxychloroquine and chloroquine, azathioprine, glucocorticoids and non-steroidal anti-inflammatories (NSAIDs), e.g. cyclooxygenase inhibitors, selective COX-2 inhibitors, agents which modulate migration of immune cells, e.g. chemokine receptor antagonists, immunosuppressive or immunomodulating agents, anti-inflammatory agents, calcineurin inhibitor, e.g. cyclosporin A, ISA247 or FK 506; a mTOR inhibitor, e.g. rapamycin, 40-O-(2-hydroxyethyl)-rapamycin, CC1779, ABT578, TAFA-93, AP23573, AP23464, AP23841, biolimus-7 or biolimus-9; an ascomycin having immuno-suppressive properties, e.g. ABT-281, ASM981, etc.; corticosteroids; cyclophosphamide; azathioprene; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; immunomodulatory agents, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, e.g. natalizumab (ANTEGREN®).

1. A compound of formula (I) or a pharmaceutically acceptable salt or a pharmaceutically-acceptable and -cleavable ester, or acid addition salt thereof: 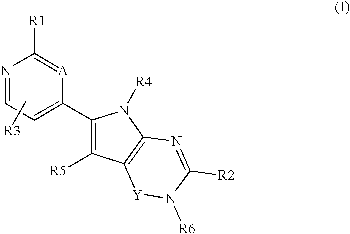
wherein A is CH or N;
Y is C═O, S═O or S(═O)₂;
R1 denotes the group —X—R¹¹;
X is a direct bond or is selected from the group consisting of C₁-C₆ alkyl, C₂-C₆ alkenyl, C₂-C₆ alkynyl, amino, aminocarbonyl, oxy, carbonyl, carboxy; carboxamido, sulfonamido, aminosulfonyl, diazo, mercapto, —CH═N—N—, —CH═N—N—CO—, —CH═N—N—CO—N—;
R¹¹ is selected from the group consisting of optionally substituted (C₁-C₆ alkyl, aryl, C₃-C₁₂ cycloalkyl, heteroaryl, heterocycloalkyl);
the optional substituent or substituents on R¹¹ being independents selected from the following: nitro, cyano, halo, hydroxyl, further optionally substituted (aryl, cycloalkyl, heteroaryl, heterocycloalkyl, aryl-C₁-C₆ alkyl, heteroaryl-C₁-C₆ alkyl, heterocycloalkyl-C₁-C₆ alkyl, cycloalkyl-C₁-C₆ alkyl, C₁-C₆ alkyl, C₂-C₆ alkenyl, C₂-C₆ alkynyl, C₁-C₆ alkyloxy, amino, carbamoyl, C₁-C₆ alkoxy, oxy, carboxy, mercapto, carboxamido, sulfonyl, sulfonamido);
such further optional substituents being selected from the group consisting of C₁-C₆ alkyl, cycloalkyl, heterocycloalkyl, aryl, heteroaryl, halo, cyano, nitro, alkoxy, hydroxyl, further optional substituted (amino, oxy, carboxy, mercapto, carboxy, carboxamido, sulfonyl, sulfonamide, alkanoyloxy;
further optional substituents being selected from the group consisting of C₁-C₆ alkyl, aryl, heteroaryl, halo, cyano, nitro, alkoxy, amino, alkylamino, dialkylamino, carboxyl, C₁-C₆ alkylcarboxyl;
R2 is selected from the group consisting of H, halo, cyano, optionally substituted (C₁-C₆ alkyl, C₂-C₆ alkenyl, C₃-C₁₂ cycloalkyl, aryl, heteroaryl, amino, mercapto, alkoxy);
the optional substituents on R2 being selected from C₁-C₆ alkyl, cycloalkyl, carboxy, sulfonyl, halo, cyano, hydroxy, alkoxy, oxy and amino;
R3 is selected from the group consisting of H, optionally substituted (C₁-C₆ alkyl, amino, alkoxy), halo, cyano and hydroxyl, optional substituents being halo, hydroxyl, alkoxy, C₁-C₆ alkyl or an amino group;
R4 is selected from the group consisting of H, optionally substituted C₁-C₆ alkyl;
the optional substituent on R4 being independently selected from: halo, cyano, C₁-C₆ alkyl, amino, alkylamino, dialkylamino, hydroxyl, alkoxy, carboxy, carboxamido;
R5 is selected from the group consisting of H, halo, cyano, optionally substituted (C₁-C₆ alkyl, amino, alkoxy);
wherein the optional substituent is/are independently selected from the list as defined for R4;
R6 is selected from the group consisting of H or optionally substituted (C₁-C₄ alkyl or C₂-C₄ alkenyl) wherein the optional substituent or substituents are independently selected from one or more of the following: halo, CN, OH, OR, NHR, NR₂, SO₂NHR, SO₂NR₂, CO₂H, CO₂R, CONHR, CONH₂, CONR₂, PO₃H₂, PO₃R₂; R denoting a C₁-C₆ alkyl group.

2. A compound of formula (II) or a pharmaceutically acceptable salt or a pharmaceutically-acceptable and -cleavable ester, or acid addition salt thereof: 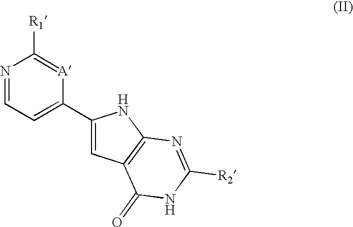
wherein A′ is CH or N;
R_{1′} denotes the group-X′—R^{11′};
X is a direct bond or is selected from the group consisting of C₁-C₆ alkyl, C₂-C₆ alkenyl, C₂-C₆ alkynyl, amino, aminocarbonyl, oxy, carbonyl, carboxy; carboxamido, sulfonamido, aminosulfonyl, diazo, mercapto, —CH═N—N—, —CH═N—N—CO—, —CH═N—N—CO—N—;
R¹¹ is selected from the group consisting of optionally substituted (C₁-C₆ alkyl, aryl, C₃-C₁₂ cycloalkyl, heteroaryl, heterocycloalkyl);
the optional substituent or substituents on R¹¹ being independents selected from the following: nitro, cyano, hydroxyl, halo, further optionally substituted (aryl, cycloalkyl, heteroaryl, heterocycloalkyl, C₁-C₆ alkyl, C₂-C₆ alkenyl, C₂-C₆ alkynyl, C₁-C₆ alkyloxy, amino, oxy, carboxy, mercapto, carboxy, carboxamido, sulfonyl, sulfonamido);
such further optional substituents being selected from the group consisting of C₁-C₆ alkyl, cycloalkyl, heterocycloalkyl, halo, cyano, hydroxyl, nitro, alkoxy, further optional substituted (amino, oxy, carboxy, mercapto, carboxy, carboxamido, sulfonyl, sulfonamido); the further optional substituents being as defined above with respect to R¹¹.

3. A compound of formula (III) or a pharmaceutically acceptable salt or a pharmaceutically-acceptable and -cleavable ester, or acid addition salt thereof: 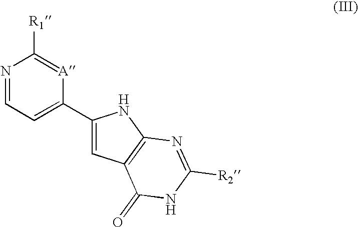
wherein A″ is CH or N;
R₁″ is selected from the following: 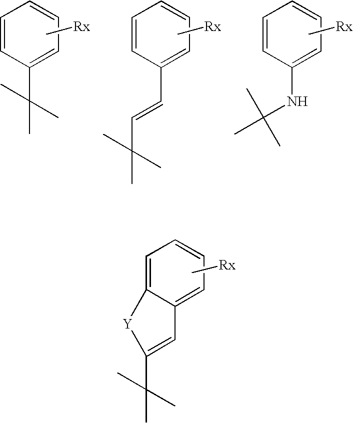
wherein:
Y is O, N, S or —C═N—;
Rx is selected from optionally substituted (aryl, cycloalkyl, heteroaryl, heterocycloalkyl, C₁-C₆ alkyl, C₂-C₆ alkenyl, C₂-C₆ alkynyl, C₁-C₆ alkyloxy, amino, oxy, carboxy, mercapto, carboxamido, sulfonyl, sulfonamido), hydroxyl, halo, nitro, cyano;
the optional substituents on Rx being selected from the group consisting of C₁-C₆ alkyl, cycloalkyl, heterocycloalkyl, halo, cyano, hydroxyl, amino, alkylamino, dialkylamino, carboxy, carboxamido, sulfonamido;
R₂″ is selected from H, halo, cyano, optionally substituted (C₁-C₆ alkyl, C₂-C₆ alkenyl, C₃-C₁₂ cycloalkyl, aryl, heteroaryl, mercapto, alkoxy, amino); the optional substituents being as defined above for Rx.

4. A compound according to claim 1 selected from the following:
6-[2-((E)-Styryl)-pyridin-4-yl]-3,7-dihydro-pyrrolo[2,3-d]pyrimidin-4-one
6-[2-((E)-Styryl)-pyridin-4-yl]-2-trifluoromethyl-3,7-dihydro-pyrrolo[2,3-d]pyrimidin-4-one
2-Methyl-6-[2-((E)-styryl)-pyridin-4-yl]-3,7-dihydro-pyrrolo[2,3-d]pyrimidin-4-one
2-Amino-6-[2-((E)-styryl)-pyridin-4-yl]-3,7-dihydro-pyrrolo[2,3-d]pyrimidin-4-one
6-[2-(4-Fluoro-phenyl)-pyridin-4-yl]-3,7-dihydro-pyrrolo[2,3-d]pyrimidin-4-one
6-[2-(3-Fluoro-phenyl)-pyridin-4-yl]-3,7-dihydro-pyrrolo[2,3-d]pyrimidin-4-one
6-[2-(3-Amino-phenyl)-pyridin-4-yl]-3,7-dihydro-pyrrolo[2,3-d]pyrimidin-4-one
6-[2-(3-Fluoro-4-methoxy-phenyl)-pyridin-4-yl]-3,7-dihydro-pyrrolo[2,3-d]pyrimidin-4-one
6-(6′-Methoxy-[2,3′]bipyridinyl-4-yl)-3,7-dihydro-pyrrolo[2,3-d]pyrimidin-4-one
6-[2-(4-Morpholin-4-yl-phenyl)-pyridin-4-yl]-3,7-dihydro-pyrrolo[2,3-d]pyrimidin-4-one
6-[2-(4-Morpholin-4-ylmethyl-phenyl)-pyridin-4-yl]-3,7-dihydro-pyrrolo[2,3-d]pyrimidin-4-one
6-{2-[(E)-2-(4-Fluoro-phenyl)-vinyl]-pyridin-4-yl}-3,7-dihydro-pyrrolo[2,3-d]pyrimidin-4-one
6-(2-Benzofuran-2-yl-pyridin-4-yl)-3,7-dihydro-pyrrolo[2,3-d]pyrimidin-4-one
6-{2-[(E)-2-(4-Dimethylamino-phenyl)-vinyl]-pyridin-4-yl}-3,7-dihydro-pyrrolo[2,3-d]pyrimidin-4-one
2-Amino-6-{2-[(E)-2-(4-dimethylamino-phenyl)-vinyl]-pyridin-4-yl}-3,7-dihydro-pyrrolo[2,3-d]pyrimidin-4-one
2-Amino-6-{2-[(E)-2-(4-fluoro-phenyl)-vinyl]-pyridin-4-yl}-3,7-dihydro-pyrrolo[2,3-d]pyrimidin-4-one
6-(2-Benzo[b]thiophen-2-yl-pyridin-4-yl)-3,7-dihydro-pyrrolo[2,3-d]pyrimidin-4-one
6-(2-Quinolin-3-yl-pyridin-4-yl)-3,7-dihydro-pyrrolo[2,3-d]pyrimidin-4-one
6-[2-(1H-Indol-2-yl)-pyridin-4-yl]-3,7-dihydro-pyrrolo[2,3-d]pyrimidin-4-one
6-(2-{(E)-2-[4-(4-Methyl-piperazin-1-ylmethyl)-phenyl]-vinyl}-pyridin-4-yl)-3,7-dihydro-pyrrolo[2,3-d]pyrimidine-one
6-{2-[(E)-2-(4-Diethylaminomethyl-phenyl)-vinyl]-pyridin-4-yl}-3,7-dihydro-pyrrolo[2,3-d]pyrimidin-4-one
2,2-Dimethyl-propionic acid 4-{(E)-2-[4-(4-oxo-4,7-dihydro-3H-pyrrolo[2,3-d]pyrimidin-6yl)-pyridin-2-yl]-vinyl}-benzyl ester
2,2-Dimethyl-propionic acid 2-{(E)-2-[4-(4-oxo-4,7-dihydro-3H-pyrrolo[2,3-d]pyrimidin-6yl)-pyridin-2-yl]-vinyl}-benzyl ester
6-{2-[(E)-2-(4-Piperidin-1-ylmethyl-phenyl)-vinyl]-pyridin-4-yl}-3,7-dihydro-pyrrolo[2,3-d]pyrimidin-4-one
4-(4-{(E)-2-[4-(4-Oxo-4,7-dihydro-3H-pyrrolo[2,3-d]pyrimidin-6-yl)-pyridin-2-yl]-vinyl}-benzyl)-piperazine-1-carboxylic acid tert.-butyl ester
6-{2-[(E)-2-(3-Fluoro-4-morpholin-4-ylmethyl-phenyl)-vinyl]-pyridin-4-yl}-3,7-dihydro-pyrrolo[2,3-d]pyrimidin-4-one
6-{2-[-2-(3-Fluoro-4-morpholin-4-ylmethyl-phenyl)-ethyl]-pyridin-4-yl}-3,7-dihydro-pyrrolo[2,3-d]pyrimidin-4-one
6-{2-[(E)-2-(3-Morpholin-4-ylmethyl-phenyl)-vinyl]-pyridin-4-yl}-3,7-dihydro-pyrrolo[2,3-d]pyrimidin-4-one
6-(2-{(E)-2-[4-(2-Morpholin-4-yl-2-oxo-ethyl)-phenyl]-vinyl}-pyridin-4-yl)-3,7-dihydro-pyrrolo[2,3-d]pyrimidin-4-one
6-[2-(4-Morpholin-4-ylmethyl-phenylethynyl)-pyridin-4-yl]-3,7-dihydro-pyrrolo[2,3-d]pyrimidin-4-one
6-{2-[2-(4-Morpholin-4-ylmethyl-phenyl)-ethyl]-pyridin-4-yl}-3,7-dihydro-pyrrolo[2,3-d]pyrimidine-4-one
6-{2-[(E)-2-(4-Morpholin-4-ylmethyl-phenyl)-vinyl]-pyridin-4-yl}-3,7-dihydro-pyrrolo[2,3-d]pyrimidin-4-one
N,N-Diethyl-4-{(E)-2-[4-(4-oxo-4,7-dihydro-3H-pyrrolo[2,3-d]pyrimidin-6-yl)-pyridin-2-yl]-vinyl}benzamide
6-(2-{(E)-2-[4-(Morpholine-4-carbonyl)-phenyl]-vinyl}-pyridin-4-yl)-3,7-dihydro-pyrrolo[2,3-d]pyrimidin-4-one
6-(2-{(E)-2-[4-(4-Hydroxy-piperidine-1-carbonyl)-phenyl]-vinyl}-pyridin-4-yl)-3,7-dihydro-pyrrolo[2,3-d]pyrimidin-4-one
6-(2-{(E)-2-[3-(Morpholine-4-carbonyl)-phenyl]-vinyl}-pyridin-4-yl)-3,7-dihydro-pyrrolo[2,3-d]pyrimidin-4-one
6-[2-((E)-2-Pyridin 3-yl-vinyl)-pyridin-4-yl]-3,7-dihydro-pyrrolo[2,3-d]pyrimidin-4-one
6-(2-{(E)-2-[4-(4-Acetyl-piperazin-1-ylmethyl)-phenyl]-vinyl}-pyridin-4-yl)-3,7-dihydro-pyrrolo[2,3-d]pyrimidin-4-one
2-Amino-6-{2-[(E)-2-(4-morpholin-4-ylmethyl-phenyl)-vinyl]-pyridin-4-yl}-3,7-dihydro-pyrrolo[2,3-d]pyrimidin-4-one
6-{2-[(E)-2-(4-Morpholin-4-ylmethyl-phenyl)-vinyl]-pyridin-4-yl}-2-trifluoromethyl-3,7-dihydro-pyrrolo[2,3-d]pyrimidin-4-one
2-Methyl-6-{2-[(E)-2-(4-morpholin-4-ylmethyl-phenyl)-vinyl]-pyridin-4-yl}-3,7-dihydro-pyrrolo[2,3-d]pyrimidin-4-one
6-{2-[(E)-2-(4-Hydroxyphenyl)-vinyl]-pyridin-4-yl}-3,7-dihydro-pyrrolo[2,3-d]pyrimidin-4-one
6-[2-((E)-2-Cyclohexyl-vinyl)-pyridin-4-yl]-3,7-dihydro-pyrrolo[2,3-d]pyrimidin-4-one
6-(2-{(E)-2-[4-(2-Dimethylamino-ethoxy)-phenyl]-vinyl}-pyridin-4-yl)-3,7-dihydro-pyrrolo[2,3-d]pyrimidin-4-one
6-(2-{(E)-2-[4-(2-Morpholin-4-yl-ethoxy)-phenyl]-vinyl}-pyridin-4-yl)-3,7-dihydro-pyrrolo[2,3-d]pyrimidin-4-one
6-(2-{(E)-2-[4-(2-Hydroxy-2-methyl-propoxy)-phenyl]-vinyl}-pyridin-4-yl)-3,7-dihydro-pyrrolo[2,3-d]pyrimidin-4-one
6-[6′-(4-Methyl-piperazin-1-yl)-[2,3′]bipyridinyl-4-yl]-3,7-dihydro-pyrrolo[2,3-d]pyrimidin-4-one
2-Amino-6-[6′-(4-methyl-piperazin-1-yl)-[2,3′]bipyridinyl-4-yl]-3,7-dihydro-pyrrolo[2,3-d]pyrimidin-4-one
6-(6′-Pyrrolidin-1-yl-[2,3′]bipyridinyl-4-yl)-3,7-dihydro-pyrrolo[2,3-d]pyrimidin-4-one
6-[6′-(2-Pyrrolidin-1-yl-ethoxy)-[2,3′]bipyridinyl-4-yl]-3,7-dihydro-pyrrolo[2,3-d]pyrimidin-4-one
2-Benzyl-6-[2-((E)-styryl)-pyridin-4-yl]-3,7-dihydro-pyrrolo[2,3-d]pyrimidin-4-one
2-Butyl-6-[2-((E)-styryl)-pyridin-4-yl]-3,7-dihydro-pyrrolo[2,3-d]pyrimidin-4-one
2-Cyclopropyl-6-[2-((E)-styryl)-pyridin-4-yl]-3,7-dihydro-pyrrolo[2,3d]pyrimidin-4-one
2-Methylsulfanyl-6-[2-((E)-styryl)-pyridin-4-yl]-3,7-dihydro-pyrrolo[2,3-d]pyrimidin-4-one
2-(2,3-Dihydroxy-propylamino)-6-[2-((E)-styryl)-pyridin-4-yl]-3,7-dihydro-pyrrolo[2,3-d]pyrimidin-4-one
2-(2-Hydroxy-1-hydroxymethyl-ethylamino)-6-[2-((E)-styryl)-pyridin-4-yl]-3,7-dihydro-pyrrolo[2,3-d]pyrimidin-4-one
6-[2-(4-Benzyloxy-phenyl)-pyridin-4-yl]-3,7-dihydro-pyrrolo[2,3-d]pyrimidin-4-one
N-Cyclopentyl-4-[4-(4-oxo-4,7-dihydro-3H-pyrrolo[2,3-d]pyrimidin-6-yl)-pyridin-2-yl]-benzamide
N-(4-Hydroxy-cyclohexyl)-4-[4-(4-oxo-4,7-dihydro-3H-pyrrolo[2,3-d]pyrimidin-6-yl)-pyridin-2-yl]-benzamide
N-[2-(2-Methoxy-ethoxy)-ethyl]-4-[4-(4-oxo-4,7-dihydro-3H-pyrrolo[2,3-d]pyrimidin-6-yl)-pyridin-2-yl]-benzamide
2-(3-Methyl-butylamino)-6-[2-((E)-styryl)-pyridin-4-yl]-3,7-dihydro-pyrrolo[2,3-d]pyrimidin-4-one
2-(2-Hydroxy-ethylamino)-6-[2-((E)-styryl)-pyridin-4-yl]-3,7-dihydro-pyrrolo[2,3-d]pyrimidin-4-one
3-(2-Methoxy-ethyl)-6-[2-((E)-styryl)-pyridin-4-yl]-3,7-dihydro-pyrrolo[2,3-d]pyrimidin-4-one.

5. A compound of claim 1 wherein the compound is an acid addition salt.

6-9. (canceled)

10. A process for preparing a compound of formula (I) in free or salt form, comprising the step of:
(i) reacting a compound of formula X with a compound of formula XIa or XIb: 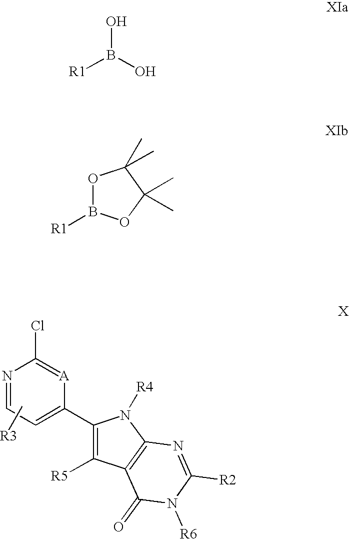
in the presence of a suitable catalyst, a base and solvent; R1-R6, A and Y being as defined above with respect to formula (I); or
(ii) for compounds of formula (I) wherein R1 is a substituted amino group having the formula R¹¹—NH—, by reacting a compound of formula X with a compound of formula XIII: 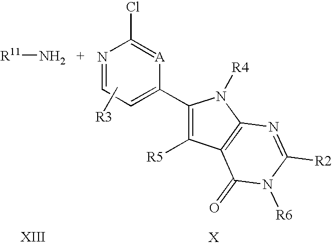
Using a suitable catalyst in the presence of a base and solvent; or
(iii) for compounds of formula (I) wherein A is N and R1 is denoted by R¹¹—X— wherein X is a direct bond, by reacting any compound of formula XV with a suitable organometallic reagent R¹¹-M: 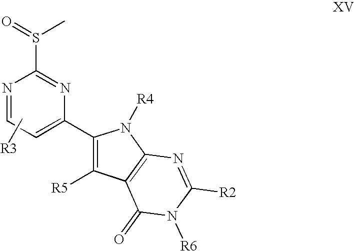
using a suitable anhydrous solvent; or
(iv) for compounds of formula I wherein A is N and R1 is R¹¹—NH—, by reacting a compound of formula XV as shown above with a compound of formula R¹¹—NH₂ wherein R11 is defined above with respect to formula (I), in the presence of a base and a suitable solvent.

11. A combination comprising a compound according to claim 1 and an active compound selected from an anti IL-1 agent, anti cytokine and anti-cytokine receptor agent, B-cell and T-cell modulating drugs, disease-modifying anti-rheumatic agents (DMARDs), gold salts, penicillamine, hydroxychloroquine, chloroquine, azathioprine, glucocorticoids, non-steroidal anti-inflammatories (NSAIDs), selective COX-2 inhibitors, chemokine receptor antagonists, modulators of adhesion molecules, immunosuppressive or immunomodulating agents, anti-inflammatory agents, calcineurin inhibitor, mTOR inhibitor, corticosteroids; PKC inhibitor, S1P receptor agonist or modulator, immunosuppressive monoclonal antibodies, immunomodulatory agents, adhesion molecule inhibitors, VCAM-4 antagonists or VLA-4 antagonists for simultaneous, separate or sequential use.

12. A method of treatment of cytokine mediated conditions comprising administering an effective amount of a compound of claim 1 or a pharmaceutically-acceptable and -cleavable ester, or acid addition salt thereof to a patient in need of such treatment.

13. A method of treatment of cytokine mediated conditions comprising administering an effective amount of a compound of claim 2 or a pharmaceutically-acceptable and -cleavable ester, or acid addition salt thereof to a patient in need of such treatment.

14. A method of treatment of cytokine mediated conditions comprising administering an effective amount of a compound of claim 3 or a pharmaceutically-acceptable and -cleavable ester, or acid addition salt thereof to a patient in need of such treatment.

15. A pharmaceutical composition comprising a compound of claim 1 or a pharmaceutically-acceptable and -cleavable ester, or acid addition salt thereof in association with a pharmaceutically acceptable excipient, diluent or carrier.

16. A pharmaceutical composition comprising a compound of claim 2 or a pharmaceutically-acceptable and -cleavable ester, or acid addition salt thereof in association with a pharmaceutically acceptable excipient, diluent or carrier.

17. A pharmaceutical composition comprising a compound of claim 3 or a pharmaceutically-acceptable and -cleavable ester, or acid addition salt thereof in association with a pharmaceutically acceptable excipient, diluent or carrier.
